(19) United States
(12) Patent Application Publication
(10) Pub. No.: US 2026/0265833 A1
(43) Pub. Date: Sep. 10, 2026

(54) METHODS FOR TREATING AND MONITORING CANCER

(71) Applicant: GLAXOSMITHKLINE INTELLECTUAL PROPERTY (NO.4) LIMITED, Stevenage (GB)

(72) Inventors: Qu ZHANG, Kirkland, WA (US); Bin FENG, Waltham, MA (US); Michael WASHBURN, Collegeville, PA (US); Sammy YUAN, Collegeville, PA (US); Racha CHEIKH AL GHANAMI, Mississauga (CA)

(73) Assignee: GLAXOSMITHKLINE INTELLECTUAL PROPERTY (NO.4) LIMITED, Stevenage (GB)

(21) Appl. No.: 19/469,691

(22) PCT Filed: Apr. 4, 2024
(86) PCT No.: PCT/US2024/023044

§ 371 Date: Sep. 26, 2025

Related U.S. Application Data
(60) Provisional application No. 63/494,595, filed on Apr. 6, 2023.
(60) Provisional application No. 63/571,064, filed on Mar. 28, 2024.

(51) Int. Cl. C12Q 1/6886; A61K 39/00; A61P 35/00; C07K 16/28

(52) U.S. Cl. CPC C12Q 1/6886; A61P 35/00; C07K 16/2818; A61K 2039/505; C07K 2317/24; C12Q 2600/154; C12Q 2600/156

(57) ABSTRACT

The disclosure provides a method of treating cancer in a patient by the administration of certain immune check point inhibitors and monitoring molecular response.

### TECHNICAL FIELD

This disclosure relates to methods for treating cancer and methods for monitoring the response of a cancer to treatments and in particular the response of a cancer to immune checkpoint inhibitors.

### BACKGROUND

Circulating tumor DNA (ctDNA) is a promising non-invasive oncology biomarker generally believed to be correlated with long term clinical outcome. Despite this belief, confounding clinical evidence has limited the systematic use of ctDNA as a clinical trial endpoint. Typically, clinical trial patient ctDNA has been evaluated in post clinical trial retrospective analyses or merely considered as additional descriptive patient data.

Consequently, there exists an unmet medical need for improved methods for treating cancer; incorporating improved methods of using ctDNA particularly in conjunction with immune checkpoint inhibitors; and, improved methods for monitoring the response to such treatments.

### SUMMARY

We provide improved methods for treating cancer and monitoring clinical response with ctDNA. In particular, a method for treating a solid tumor in a human patient, the method comprising administering an immune checkpoint inhibitor to the patient having circulating tumor DNA that is detectable before the first dose of the immune checkpoint inhibitor and a level of circulating tumor DNA is decreased following one or more doses of the immune checkpoint inhibitor by at least from about 5% to about 70%.

In one aspect, the disclosure provides a method of treating cancer in a patient comprising providing an immune checkpoint inhibitor to the patient; detecting circulating tumor DNA in the patient; determining a molecular response rate of the cancer to the immune checkpoint inhibitor; and, determining a radiologic objective response rate.

In one aspect, the disclosure provides a method of treating cancer in a patient comprising determining a baseline circulating tumor DNA profile from a first liquid biopsy; providing an immune checkpoint inhibitor to the patient; determining a second circulating tumor DNA level from a second liquid biopsy; determining a molecular response rate of the cancer to the immune checkpoint inhibitor based on the step of determining a baseline and the step of determining a second circulating tumor DNA level; and, determining a radiologic objective response rate.

In one aspect, the disclosure provides a method of treatment of cancer in a patient previously treated with an immune checkpoint inhibitor comprising the steps of detecting circulating tumor DNA in a liquid biopsy from the patient, determining a molecular response rate, and treating the patient with an immune checkpoint inhibitor.

In one aspect, the disclosure provides a method of treatment of cancer comprising dosing a patient with an immune checkpoint inhibitor, detecting circulating tumor DNA after a therapeutic interval, and determining on-treatment change in cancer. In some embodiments the therapeutic interval is 3, 4, 5, 6, 7, 8, 9, 10, 11, 12, 13, 14, 15, 16, 17, 18, 19, 20, 21, 22, 23, or 24 weeks after the first immune checkpoint inhibitor dose.

In some embodiments, the cancer is non-small cell lung cancer. In some embodiments, the cancer is a solid tumor. In some embodiments, the cancer is MSI-H, dMMR, or has a high TMB. In some embodiments, the immune checkpoint inhibitor is an anti-PD-1 inhibitor, an anti-PD-L1 inhibitor, a CTLA-4 inhibitor, a TIGIT inhibitor, a TIM-3 inhibitor, or combinations thereof.

The disclosure provides a method of treating cancer in a patient comprising providing an immune checkpoint inhibitor to the patient; detecting circulating tumor DNA in the patient; determining a molecular response rate of the cancer to the immune checkpoint inhibitor; and, determining a radiologic objective response rate. In another aspect, the disclosure provides a method of treating cancer in a patient comprising determining a baseline circulating tumor DNA profile from a first liquid biopsy; providing an immune checkpoint inhibitor to the patient; determining a second circulating tumor DNA level from a second liquid biopsy; determining a molecular response rate of the cancer to the immune checkpoint inhibitor based on the step of determining a ctDNA baseline and the step of determining a second circulating tumor DNA level; and, determining a radiologic objective response rate.

In one aspect, the disclosure provides a method of treatment of cancer in a patient previously treated with an immune checkpoint inhibitor comprising the steps of detecting circulating tumor DNA in a liquid biopsy from the patient, determining a molecular response rate, and treating the patient with an immune checkpoint inhibitor.

In one aspect, the disclosure provides a method of treatment of non small cell lung cancer comprising dosing a patient with an immune checkpoint inhibitor, detecting circulating tumor DNA after a therapeutic interval, and determining on-treatment change in cancer. In some embodiments the therapeutic interval is 3, 4, 5, 6, 7, 8, 9, 10, 11, 12, 13, 14, 15, 16, 17, 18, 19, 20, 21, 22, 23, or 24 weeks after the first immune checkpoint inhibitor dose.

In one aspect, the disclosure provides a method for treating a solid tumor in a human patient, the method comprising administering an immune checkpoint inhibitor to the patient having circulating tumor DNA that is detectable before the first dose of the immune checkpoint inhibitor and a level of circulating tumor DNA is decreased following one or more doses of the immune checkpoint inhibitor by at least from about 5% to about 70%. In some embodiments the level of circulating tumor DNA is decreased following one or more doses of the immune checkpoint inhibitor as compared to the circulating tumor DNA that is detectable before the first dose of the immune checkpoint inhibitor by at least from about 5% to about 10%; from about 5% to about 15%; from about 5% to about 20%; from about 5% to about 25%; from about 5% to about 30%; from about 5% to about 35%; from about 5% to about 40%; from about 5% to about 45%; from about 5% to about 50%; at least 50%; at least more than 50% to about 60%; at least more than 50% to about 65%; at least more than 50% to about 70%; more than 70%; about 75%; about 75% to about 100%.

In some embodiments, the immune checkpoint inhibitor is an anti-PD-1 inhibitor, an anti-PD-L1 inhibitor, a CTLA-4 inhibitor, a TIGIT inhibitor, a TIM-3 inhibitor, or a combination thereof. In some embodiments, the immune checkpoint inhibitor is pembrolizumab, nivolumab, cemiplimab, ipilimumab, dostarlimab, or a biosimilar thereof. In some embodiments, the immune checkpoint inhibitor is a combination of an anti-PD-1 inhibitor and a TIGIT inhibitor. In some embodiments, the anti-PD-1 inhibitor is selected from the group consisting of dostarlimab and pembrolizumab and the TIGIT inhibitor is selected from the group consisting of vibostolimab and EOS-448.

It is understood that any and all embodiments of the present disclosure may be taken in combination with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### BRIEF DESCRIPTION OF THE DRAWINGS

FIG. 1: Molecular Response Rate (MRR) significantly correlated with Objective Response Rate (ORR) in Immune Checkpoint Inhibitor (ICI) trials.

FIG. 2: A framework to assess a correlation between an MRR difference (AMRR) and OS HR.

FIG. 3: Digitization of published MYSTIC ctDNA MR data to retrieve patient level clinical outcome.

FIG. 4: AMRR inversely correlated with OS hazard ratio in simulations using digitized MYSTIC data.

FIG. 5: An inverse correlation between a AMRR and an OS hazard correlated in the dataset with an association between a MR and an OS.

FIG. 6: A schematic of the patient plasma sample collection timings for the study described in Example 3.

FIG. 7A: shows ctDNA detection at specific points of time over the duration of the study. An increasing trend can be seen from pre-operative stage to follow-ups 3-6 stage.

FIG. 7B: shows assay success at specific points of time over the duration of the study as a measure of the proportion of patients in the study.

FIG. 8: An oncoplot showing the clinical features of individual patients categorized by estrogen receptor subtype (ER+) or triple negative breast cancer (TNBC). These clinical variables include baseline ctDNA detection, disease recurrence, and pathologic complete response (pCR).

FIG. 9A: shows two box plots showing ctDNA detection compared to the baseline detection outcome. The box represents the 25th to 75th percentiles, while the whiskers represent the minimum and maximum values.

FIG. 9B: shows a bar graph showing the proportion of baseline study samples with ctDNA detection categorized by clinical variables. These clinical variables include age, post or pre-menopausal status, prior treatment (taxane or anthracycline and taxane), pathological complete response, disease stage, nodal stage, and recurrences.

FIG. 9C-9J: shows box plots of quantitative methylation scores and their association to clinical variables of interest. The box represents the 25th to 75th percentiles, while the whiskers represent the minimum and maximum values. FIG. 4C depicts quantitative methylation scores associated with post and pre-menopausal status. FIG. 4D depicts quantitative methylation scores associated with receptor subtype ER+ and TNBC. FIG. 4E depicts quantitative methylation scores associated with pCR. FIG. 4F depicts quantitative methylation scores associated with RCB-0/1 and RCB-2/3. FIG. 4G depicts quantitative methylation scores associated with disease stage. FIG. 4H depicts quantitative methylation scores associated with nodal stage. FIG. 4I depicts quantitative methylation scores associated with grade. FIG. 4J depicts quantitative methylation scores associated with recurrence.

FIG. 10A: Swimmer plot showing for individual subjects ctDNA detection in relation to each estrogen receptor positive (ER+) participant's clinical timeline.

FIG. 10B: Swimmer plot showing for individual subjects ctDNA detection in relation to each triple negative breast cancer (TNBC) participant's clinical timeline.

FIG. 11A: shows the exploratory outcome for subjects with ctDNA detection at baseline.

FIG. 11B: shows the exploratory outcome for subjects with post operative ctDNA detection.

FIG. 11C: shows the exploratory outcome for subjects with ctDNA detection at any post operative or later follow up timepoint.

### DEFINITIONS

“Antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long): an amino-terminal variable (VH) domain (located at the tips of the Y structure), in followed by three constant domains: CH1, CH2, and the carboxy-terminal CH3 (located at the base of the Y's stem). A short region, known as the “switch,” connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains: an amino-terminal variable (VL) domain, followed by a carboxy-terminal constant (CL) domain, separated from one another by another “switch.” Those skilled in the art are well familiar with antibody structure and sequence elements, recognize “variable” and “constant” regions in provided sequences, and understand that there may be some flexibility in definition of a “boundary” between such domains such that different presentations of the same antibody chain sequence may, for example, indicate such a boundary at a location that is shifted one or a few residues relative to a different presentation of the same antibody chain sequence. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered glycosylation. For purposes of the present disclosure, in certain embodiments, any polypeptide or complex of polypeptides that includes sufficient immunoglobulin domain sequences as found in natural antibodies can be referred to and/or used as an “antibody,” whether such polypeptide is naturally produced (e.g., generated by an organism reacting to an antigen), or produced by recombinant engineering, chemical synthesis, or other artificial system or methodology. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, fully human, and the like, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi- or multi-specific antibodies; antibody fragments such as Fab fragments, Fab′ fragments, F(ab′)2 fragments, Fd′ fragments, Fd fragments, and isolated CDRs or sets thereof; single chain Fvs; polypeptide-Fc fusions; and single domain antibodies. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, and the like), or other pendant group (e.g., poly-ethylene glycol, and the like)).

“ctDNA” refers to circulating tumor DNA and is the portion of cell-free DNA (cfDNA) derived from cancer (tumor) cells. The majority of ctDNA is produced by apoptosis has a size of approximately 166 bp, which corresponds to 147 bp of DNA wrapped around a nucleosome plus the DNA on ligand-histone H1. Therefore, typically, ctDNA comprises strands of <145 bp in length and may be highly fragmented. ctDNA is obtained from a variety of patient samples, including plasma, urine, stool, saliva, seminal fluid, pleural fluid, cerebrospinal fluid, and peritoneal fluid. ctDNA molecules may occur as DNA fragments. Regardless of specific source, these patient samples are referred to as liquid biopsies.

“Methylation” or “DNA methylation” refers to addition of a methyl group to a nucleobase in a nucleic acid molecule. In some embodiments, methylation refers to addition of a methyl group to a cytosine at a CpG site (cytosine-phosphate-guanine site (i.e., a cytosine followed by a guanine in a 5′-3′ direction of the nucleic acid sequence). In some embodiments, DNA methylation refers to addition of a methyl group to adenine, such as in N6-methyladenine. In some embodiments, DNA methylation is 5-methylation (modification of the 5th carbon of the 6-carbon ring of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

“Hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues. Hypermethylated promoter DNA regions may be indicative of cancer.

“Molecular Response” or “MR” refers to the detection of a response to a treatment by measuring a biomarker, ctDNA level, change in levels of a methylation level of one or more loci, or some other laboratory-measured value. “MR Clearance” refers to the change in tumor status following a line of therapy where the particular biomarker, ctDNA allele, or bulk ctDNA levels, falls at or below the lower limit of detection for the laboratory test. In some embodiments, the ctDNA level is determined as the mean variant allele fraction (VAF) from a particular patient sample and the Molecular Response (MR) the ratio of the mean VAF measured during a line of therapy or treatment to the baseline VAF. A MR can be calculated at multiple time points while a patient is on treatment by collecting time sequenced liquid biopsies.

“Immune Checkpoints” refers to a group of molecules on the surface of CD4 T-cells and CD8 T-cells. These molecules comprise a cell signaling network that reduces or down-modulates the tumor directed immune response. Exemplary immune checkpoint molecules include Programmed Death-Ligand 1 (PD-L1, also known as B7-H1, CD274), Programmed Death 1 (PD-1), CTLA-4, CD137, CD40, 2B4, IDO1, IDO2, VISTA, CD27, CD28, PD-L2 (B7-DC, CD273), LAG3, CD80, CD86, PDL2, B7H3, HVEM, BTLA, KIR, GAL9, TIM-3, PVRIG, TIGIT, A2aR, MARCO (macrophage receptor with collageneous structure), PS (phosphatidylserine), CD276, VTCN1, CD70, CD96, and CD160.

“Immune checkpoint Inhibitor”, “checkpoint inhibitor” or simply “ICI” refers to any modulator that reduces or inhibits the activity of an immune checkpoint molecule. ICIs may be small molecules, antibodies, antibody-derivatives (including Fab fragments and scFvs), antibody-drug conjugates, antisense oligonucleotides, siRNA, aptamers, peptides, and peptide mimetics. Inhibitory nucleic acids that decrease the expression and/or activity of immune checkpoint molecules can also be used in the methods disclosed herein.

“Microsatellite instability (MSI)” refers to the form of genomic instability associated with defective DNA mismatch repair in tumors. See Boland et al., Cancer Research 58, 5258-5257, 1998. In one embodiment, MSI analysis can be carried out using the five National Cancer Institute (NCI) recommended microsatellite markers: BAT25 (GenBank accession no. 9834508), BAT26 (GenBank accession no. 9834505), D5S346 (GenBank accession no. 181171), D2S123 (GenBank accession no. 187953), D17S250 (GenBank accession no. 177030). Additional markers for example, BAT40, BAT34C4, TGF-β-RII and ACTC can be used. Commercially available kits for MSI analysis include, for example, the Promega MSI multiplex PCR assay, FoundationOne® CDx (F1CDx) next generation sequencing based in vitro diagnostic device using DNA isolated from formalin-fixed, paraffin-embedded (FFPE) tumor tissue specimens.

“High frequency microsatellite instability” or “microsatellite instability-high (MSI-H)” refers to if two or more of the five NCI markers indicated above show instability or ≥30-40% of the total markers demonstrate instability (i.e., have insertion/deletion mutations). “Non-MSI-H cancer” as used herein refers to microsatellite stable (MSS) and low frequency MSI (MSI-L) cancer. “Microsatellite Stable (MSS)” refers to if none of the five NCI markers indicated above show instability (i.e., have insertion/deletion mutations). “Proficient mismatch repair (pMMR) cancer” refers to normal expression of MMR proteins (MLH1, PMS2, MSH2, and MSH6) in tumor specimen by IHC. Commercially available kits for MMR analysis include the Ventana MMR IHC assay.

“Mismatch repair deficient (dMMR) cancer” refers to low expression of one or more MMR protein(s) (MLH1, PMS2, MSH2, and MSH6) in a tumor specimen by IHC. “Variable regions” or “V region” as used herein means the segment of IgG chains which is variable in sequence between different antibodies. It extends to Kabat residue 109 in the light chain and 113 in the heavy chain. The term “buffer” encompasses those agents which maintain the solution pH of the formulations of the invention in an acceptable range, or, for lyophilized formulations of the invention, provide an acceptable solution pH prior to lyophilization.

“Objective Response Rate” or “ORR” means a diagnostic imaging-based measure used in oncology clinical trials outcome measure assessed using Response Evaluation Criteria in Solid Tumors (RECIST) response categorization based on the percent change of the tumor burden before and after treatment. RECIST 1.1 criteria and the radiographic scoring scheme are detailed European Journal of Cancer 45 (2009) 228-247.

“Complete response” or “CR” is used to mean the disappearance of all or substantially all target lesions. Any pathological lymph nodes must be <10 mm in the short axis. In some embodiments, CR refers to an about 80%, 85%, 90%, 91%, 92%, 93%, 94%, 95%, 96%, 97%, 98%, 99% or 100% decrease in the sum of the diameters of the target lesions (i.e., loss of lesions), taking as reference the baseline sum diameters. In some embodiments, CR indicates that less than about 10%, 9%, 8%, 7%, 6%, 5%, 4%, 3%, 2%, 1% or less of the total lesion diameter remains after treatment. Exemplary methods for evaluating complete response are identified by RECIST guidelines. See, e.g., E. A. Eisenhauer, et al., “New response evaluation criteria in solid tumors: Revised RECIST guideline (version 1.1.),” Eur. J. of Cancer, 45:228-247 (2009).

“Partial response” or “PR” refers to a decrease in tumor progression in a subject as indicated by a decrease in the sum of the diameters of the target lesions, taking as reference the baseline sum diameters. In some embodiments, PR refers to at least a 30% decrease in the sum of diameters of target lesions, taking as reference the baseline sum diameters. Exemplary methods for evaluating partial response are identified by RECIST guidelines. See e.g., E. A. Eisenhauer, et al., “New response evaluation criteria in solid tumors: Revised RECIST guideline (version 1.1.),” Eur. J. of Cancer, 45:228-247 (2009).

“Stabilization” of tumor growth or a “stable disease” (“SD”) refers to neither sufficient shrinkage to qualify for PR nor sufficient increase to qualify for PD. In some embodiments, stabilization refers to a less than 30%, 25%, 20%, 15%, 10% or 5% change (increase or decrease) in the sum of the diameters of the target lesions, taking as reference the baseline sum diameters. Exemplary methods for evaluating stabilization of tumor growth or a stable disease are identified by RECIST guidelines. See e.g., E. A. Eisenhauer, et al., “New response evaluation criteria in solid tumors: Revised RECIST guideline (version 1.1.),” Eur. J. of Cancer, 45:228-247 (2009).

“Progression” of tumor growth or a “progressive disease” (“PD”) as used herein in reference to cancer status indicates an increase in the sum of the diameters of the target lesions (tumors). In some embodiments, progression of tumor growth refers to at least a 20% increase in the sum of diameters of target lesions, taking as reference the smallest sum on study (this includes the baseline sum if that is the smallest on study). In some embodiments, in addition to a relative increase of 20%, the sum of diameters of target lesions must also demonstrate an absolute increase of at least 5 mm. An appearance of one or more new lesions may also be factored into the determination of progression of tumor growth.

“Pharmaceutical formulation” refers to preparations which are in such form as to permit the active ingredients to be effective, and which contains no additional components which are toxic to the subjects to which the formulation would be administered. The term “formulation” and “pharmaceutical formulation” are used interchangeably throughout.

“Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

“PD-1 antagonist” and “PD-1 inhibitor” means any chemical compound or biological molecule that blocks binding of PD-ligand 1 (PD-L1) expressed on a cancer cell to PD-1 expressed on an immune cell (T cell, B cell or natural killer T (NKT) cell) and preferably also blocks binding of PD ligand 2 (PD-L2) expressed on a cancer cell to the immune-cell expressed PD-1. Alternative names or synonyms for PD-1 and its ligands include: PDCD1, PD1, CD279 and SLEB2 for PD-1; PDCDIL1, PDL1, B7H1, B7-4, CD274 and B7-H for PD-L1; and PDCDIL2, PDL2, B7-DC, Btdc and CD273 for PD-L2. Human PD-1 amino acid sequences can be found in NCBI Locus No.: NP 005009. Human PD-L1 and PD-L2 amino acid sequences can be found in NCBI Locus No.: NP 054862 and NP_079515, respectively.

Ranges provided herein include all values within a particular range described and values about an endpoint for a particular range. The figures and tables of the disclosure also describe ranges, and discrete values, which may constitute an element of any of the methods and uses disclosed herein.

It will be appreciated that the following description is intended to refer to specific examples and is not intended to define or limit the disclosure, other than in the appended claims.

### DETAILED DESCRIPTION

Unless defined otherwise, all technical and scientific terms used herein have the same meaning as commonly understood by one of ordinary skill in the art to which this disclosure pertains. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range. The figures and tables of the disclosure also describe ranges, and discrete values, which may constitute an element of any of the methods and uses disclosed herein.

### Liquid Biopsies

Liquid Biopsies are liquid samples collected from a patient. Those skilled in the art and aware of the common general knowledge are aware of the many methods to isolate cfDNA and characterize the fraction of the total cfDNA that is ctDNA. The cfDNA and ctDNA profile obtained from a patient liquid biopsy comprise a combination of both wild-type and genetically and/or epigenetically modified DNA fragments released by different tissues and different mechanisms. Analysis of ctDNA can identify tumor-specific abnormalities, for example, point mutations, loss of heterozygosity (LOH), gene amplifications, and hypermethylation of tumor suppressor gene promoter regions.

WO2010/014920 describes one well known method for detecting and quantifying ctDNA derived from a liquid biopsy, including detecting somatic mutations by BEAMing. Other methods include Droplet digital polymerase chain reaction (ddPCR), tagged-amplicon deep sequencing (TAm-Seq), cancer personalized profiling by deep sequencing (CAPPSeq), whole-genome sequencing (WGS), and whole-exome sequencing (WES). See, e.g., O'Leary B. et al., “Comparison of BEAMing and Droplet Digital PCR for Circulating Tumor DNA Analysis”. Clin Chem (2019) 65(11):1405-13.; Valpione S, Campana L. “Detection of Circulating Tumor DNA (ctDNA) by Digital Droplet Polymerase Chain Reaction (Dd-PCR) in Liquid Biopsies”. Methods Enzymol (2019) 629:1-15; Belic J. et al., “Rapid Identification of Plasma DNA Samples With Increased ctDNA Levels by a Modified FAST-SeqS Approach.” Clin Chem (2015) 61(6):838-49. Other useful methods include those disclosed in WO2018/057928 that comprise producing libraries of detected cfDNA/ctDNA.

Exemplary methods of analyzing the methylated DNA component of the recovered ctDNA are described in WO2022/073011, WO2022/073012, WO2022/140629, WO2020/069350, and US20200340063A1. Other useful methods are known to those skilled in the art.

In some embodiments, a baseline liquid biopsy is obtained from a patient and then ctDNA is isolated before a line of treatment is administered. In some embodiments, the level of ctDNA is measured and establishes the baseline ctDNA level before a line of treatment is administered. In some embodiments, one or more liquid biopsies are obtained from a patient following one or more doses of the immune checkpoint inhibitor and the overall level of ctDNA in each such sample is determined. The difference between the baseline ctDNA level and the subsequent post-immune checkpoint inhibitor treatment ctDNA levels constitute the molecular response (MR). In some embodiments, the patient has more than one somatic mutation identified in ctDNA isolated from a liquid biopsy. In some embodiments, the highest frequency (most abundant) somatic mutation present in the recovered ctDNA is used to determine the ctDNA level for a patient.

### Methods for Treating Cancers

In patients that received immune checkpoint inhibitors (ICIs), studies have shown that 50% or greater reduction of baseline ctDNA level (ctDNA molecular response or molecular response (MR)) after several cycles of treatment is significantly associated with improved progression free survival (PFS) and overall survival (OS).

A problem posed by the potential use of MR as an endpoint for clinical response is that there is an observed discordance between MR and radiologic response. That discordance hinders the potential application of MR both as an early clinical indication of response and as a clinical trial endpoint. A solution provided herein comprises methods based on a correlation between MR rate (MRR) and radiologic objective response rate (ORR) with immune checkpoint inhibitor (ICI) intervention. Moreover, a further solution is provided by methods based on the relationship between an MRR difference and a hazard ratio of long-term survival.

In some embodiments, the methods disclosed herein may be used to identify targeted therapies to treat a cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), virus-associated cancer (including but not limited to cancers associated with human papillomavirus (HPV), hepatitis B virus (HBV), hepatitis C virus (HCV), Epstein-Barr virus (EBV), human herpesvirus type 8 (HHV-8, also known as Kaposi's sarcoma herpesvirus), HIV type 1 (HIV-1), and human T cell lymphotropic virus type 1 (HTLV-1).), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

In an embodiment, the cancer is NSCLC, such as metastatic NSCLC. In an embodiment, the NSCLC is non-squamous NSCLC. In an embodiment, the NSCLC is metastatic non-squamous NSCLC. In an embodiment, the NSCLC is NSCLC without a known epidermal growth factor receptor (EGFR), anaplastic lymphoma kinase (ALK), receptor tyrosine kinase 1 (ROS 1), or proto oncogene B-raf (BRAF) V600E mutation. In an embodiment, the NSCLC is NSCLC without a genomic aberration for which an approved targeted therapy is available. In an embodiment, the NSCLC is NSCLC without a known EGFR, ALK, ROS-1, or BRAF V600E mutation or other genomic aberration for which an approved targeted therapy is available. In an embodiment, the NSCLC is metastatic NSCLC without a known EGFR, ALK, ROS-1, or BRAF V600E mutation. In an embodiment, the NSCLC is metastatic NSCLC without a known EGFR, ALK, ROS-1, or BRAF V600E mutation or other genomic aberration for which an approved targeted therapy is available. In an embodiment, the NSCLC is non-squamous NSCLC without a known EGFR, ALK, ROS-1, or BRAF V600E mutation. In an embodiment, the NSCLC is non-squamous NSCLC without a known EGFR, ALK, ROS-1, or BRAF V600E mutation or other genomic aberration for which an approved targeted therapy is available. In an embodiment, the NSCLC is metastatic non-squamous NSCLC without a known EGFR, ALK, ROS-1, or BRAF V600E mutation. In an embodiment, the NSCLC is metastatic non-squamous NSCLC without a known EGFR, ALK, ROS-1, or BRAF V600E mutation or other genomic aberration for which an approved targeted therapy is available.

In an embodiment, the cancer is NSCLC that is a large cell neuroendocrine carcinoma (LCNEC), a lung adenocarcinoma (LUAD), or a lung squamous cell carcinoma (LUSC). In an embodiment, the NSCLC is a lung adenocarcinoma (LUAD) or a lung squamous cell carcinoma (LUSC). In an embodiment, the NSCLC is of a subtype selected from acinar LUAD, LCNEC, micropapillary LUAD, mucinous LUAD, papillary LUAD, LUSC, or solid LUAD. In an embodiment, the NSCLC is of a subtype selected from acinar LUAD, micropapillary LUAD, LUSC, or solid LUAD. In an embodiment, the NSCLC is of a subtype selected from acinar LUAD, LUSC, or solid LUAD. In an embodiment, the NSCLC is of a subtype selected from LUSC or solid LUAD. In an embodiment, the NSCLC is of a subtype that is acinar LUAD. In an embodiment, the NSCLC is of a subtype that is LUSC. In an embodiment, the NSCLC is of a subtype that is solid LUAD.

In an embodiment, the cancer is selected from the group consisting of metastatic melanoma, non-small cell lung cancer, head and neck cancer, Hodgkin's lymphoma, urothelial carcinoma, gastric cancer, cervical cancer, hepatocellular carcinoma, Merkel cell carcinoma, renal cell carcinoma, small cell lung cancer, esophageal carcinoma, endometrial cancer, squamous cell carcinoma, colorectal cancer, malignant pleural mesothelioma, bladder cancer, breast cancer, small cell lung cancer, squamous cell carcinoma, and basal cell carcinoma.

In some embodiments, a patient has a cancer with microsatellite instability. In some embodiments, the microsatellite instability is considered high, wherein the instability is significantly higher than that observed in a control cell (e.g., MSI-H status). In some embodiments, the microsatellite instability is MSI-Low. In some embodiments, the microsatellite instability is microsatellite stable (e.g., MSS status). In some embodiments, a cancer with microsatellite instability is a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus). In some certain embodiments, a cancer with microsatellite instability is an anal cancer, a fallopian tube cancer, an ovarian cancer, or a lung cancer. In some certain embodiments, a patient has an endometrial cancer with microsatellite instability. In some embodiments, a patient has an endometrial cancer that is microsatellite stable (MSS).

In embodiments, a cancer is an advanced cancer. In embodiments, a cancer is a metastatic cancer. In embodiments, a cancer is a MSI-H cancer. In embodiments, a cancer is a MSS cancer. In embodiments, a cancer is a POLE-mutant cancer. In embodiments, a cancer is a POLD-mutant cancer. In embodiments, a cancer is a high TMB cancer. In embodiments, a cancer is associated with homologous recombination repair deficiency/homologous repair deficiency (“HRD”).

In embodiments, a cancer is a solid tumor. In embodiments, a solid tumor is advanced. In embodiments, a solid tumor is a metastatic solid tumor. In embodiments, a solid tumor is a MSI-H solid tumor. In embodiments, a solid tumor is a MSS solid tumor. In embodiments, a solid tumor is a POLE-mutant solid tumor. In embodiments, a solid tumor is a POLD-mutant solid tumor. In embodiments, a solid tumor is a high TMB solid tumor. In embodiments, a solid tumor is associated with homologous recombination repair deficiency/homologous repair deficiency (“HRD”).

In embodiments, a cancer is a non-endometrial cancer (e.g., a non-endometrial solid tumor). In embodiments, a non-endometrial cancer is an advanced cancer. In embodiments, a non-endometrial cancer is a metastatic cancer. In embodiments, a non-endometrial cancer is a MSI-H cancer. In embodiments, a non-endometrial cancer is a MSS cancer. In embodiments, a non-endometrial cancer is a POLE-mutant cancer. In embodiments, a non-endometrial cancer is a solid tumor (e.g., a MSS solid tumor, a MSI-H solid tumor, a POLD mutant solid tumor, or a POLE-mutant solid tumor). In embodiments, a non-endometrial cancer is a high TMB cancer. In embodiments, a non-endometrial cancer is associated with homologous recombination repair deficiency/homologous repair deficiency (“HRD”).

In embodiments, a cancer is endometrial cancer (e.g., a solid tumor). In embodiments, an endometrial cancer is an advanced cancer. In embodiments, an endometrial cancer is a metastatic cancer. In embodiments, an endometrial cancer is a MSI-H endometrial cancer. In embodiments, an endometrial cancer is a MSS endometrial cancer. In embodiments, an endometrial cancer is a POLE-mutant endometrial cancer. In embodiments, an endometrial cancer is a POLD-mutant endometrial cancer. In embodiments, an endometrial cancer is a high TMB endometrial cancer. In embodiments, an endometrial cancer is associated with homologous recombination repair deficiency/homologous repair deficiency (“HRD”).

In an aspect, the present disclosure provides a method of treating cancer in a human patient, the method comprising: (a) identifying the patient for treatment of the cancer, wherein the patient has been determined to have the cancer based on identification of one or more cell-free nucleic acid molecules that is obtained or derived from the patient; and, (b) subjecting the patient to the treatment based on the identification in (a), wherein the treatment is an immune checkpoint inhibitor. In some embodiments the one or more cell-free nucleic acid molecules that is obtained or derived from the patient are derived from a liquid biopsy. In some embodiments the liquid biopsy is serum, pleural fluid, peritoneal fluid, or stool.

In an embodiment, the immune checkpoint inhibitor is selected from the group consisting of nivolumab, pembrolizumab, atezolizumab, avelumab, durvalumab, cemiplimab, dostarlimab, vopratelimab (JTX-4014), spartalizumab (PDR001), camrelizumab (SHR1210), sintilimab (IBI308), tislelizumab (BGB-A317), toripalimab (JS 001), cobolimab, INCMGA00012 (MGA012), AMP-224, AMP-514 (MEDI0680), acrixolimab (YBL-006), KN035, cosibelimab (CK-301), AUNP12, and CA-170, BMS-986189, a macrocyclic peptide, a modified oligonuclotide and a small molecule.

In an aspect, the present disclosure provides for a use of a pharmaceutical composition comprising an immune checkpoint inhibitor for a treatment of a cancer wherein a change in an amount of circulating tumor DNA is monitored to determine a therapeutic response. In some embodiment, the use further comprises the change in the amount of circulating tumor DNA is the change between a baseline amount and an amount after at least one dose of the pharmaceutical composition comprising the immune checkpoint inhibitor. Further, the use of comprises wherein the change in the amount of circulating tumor DNA is a reduction of at least from about 5% to about 70%; at least about 15%; at least about 15% to about 50%.

In some embodiments, the change in the amount of circulating tumor DNA is the change between a baseline amount and an amount after at least one dose of the pharmaceutical composition is determined from a liquid biopsy collected 3, 4, 5, 6, 7, 8, 9, 10, 11, 12, 13, 14, 15, 16, 17, 18, 19, 20, 21, 22, 23, or 24 weeks after the first immune checkpoint inhibitor dose. In some embodiments, MR is calculated as the ratio of the mean ctDNA VAF measured during a line of therapy or treatment to the baseline ctDNA VAF.

In an aspect, the present disclosure provides a method of treating non-small cell lung cancer (NSCLC) in a human patient, the method comprising: (a) identifying the patient for treatment of the cancer, wherein the patient has been determined to have NSCLC based on identification of one or more cell-free nucleic acid molecules that is obtained or derived from the patient; and, (b) subjecting the patient to the treatment based on the identification in (a), wherein the treatment is an immune checkpoint inhibitor. In an embodiment, the immune checkpoint inhibitor is an anti-PD-1 antibody. In an embodiment, the immune checkpoint inhibitor is pembrolizumab or dostarlimab. In an embodiment the immune checkpoint inhibitor is a combination of an anti-PD-1 antibody and an anti-TIGIT antibody. In an embodiment the immune checkpoint inhibitor is a combination of an anti-PD-1 antibody and an anti-TIM-3 antibody. In some embodiments the one or more cell-free nucleic acid molecules that is obtained or derived from the patient are obtained or derived from a liquid biopsy. In some embodiments the liquid biopsy is serum, pleural fluid, peritoneal fluid, or stool. In some embodiments the liquid biopsy is pleural fluid.

In an aspect, the present disclosure provides a method of monitoring progress of a cancer of a patient, the method comprising: (a) determining a first state of the cancer of the patient based on identification of a first set of one or more cell-free nucleic acid molecules from a first plurality of cell-free nucleic acid molecules that is obtained or derived from the patient; (b) determining a second state of the cancer of the patient based on identification of a second set of one or more cell-free nucleic acid molecules from a second plurality of cell-free nucleic acid molecules that is obtained or derived from the patient, wherein the second plurality of cell-free nucleic acid molecules are obtained from the patient subsequent to obtaining the first plurality of cell-free nucleic acid molecules from the patient, wherein the second state of the cancer is determined after one or more doses of an immune checkpoint inhibitor; and (c) determining the progress of the cancer based on the first state of the cancer and the second state of the cancer, wherein the second state of the cancer comprises a reduction in the level of cell-free nucleic acid molecules. In some embodiments the first set of one or more cell-free nucleic acid molecules from a first plurality of cell-free nucleic acid molecules that is obtained or derived from the patient are obtained or derived from a liquid biopsy. In some embodiments the second set of one or more cell-free nucleic acid molecules from a first plurality of cell-free nucleic acid molecules that is obtained or derived from the patient are obtained or derived from a liquid biopsy. In some embodiments the liquid biopsy is serum, pleural fluid, peritoneal fluid, or stool.

In an aspect, the present disclosure provides a method of monitoring progress of a cancer of a patient, the method comprising: (a) determining a first state of the cancer of the patient based on identification of a first set of one or more ctDNA molecules from a first plurality of cell-free nucleic acid molecules that is obtained or derived from the patient; (b) determining a second state of the cancer of the patient based on identification of a second set of one or more ctDNA molecules from a second plurality of cell-free nucleic acid molecules that is obtained or derived from the patient, wherein the second plurality of cell-free nucleic acid molecules are obtained from the patient subsequent to obtaining the first plurality of cell-free nucleic acid molecules from the patient, wherein the second state of the cancer is determined after one or more doses of an immune checkpoint inhibitor; and (c) determining the progress of the cancer based on the first state of the cancer and the second state of the cancer, wherein the second state of the cancer comprises a reduction in the level of ctDNA molecules. In some embodiments the first state of the cancer of the patient based on identification of a first set of one or more ctDNA molecules from a first plurality of cell-free nucleic acid molecules that is obtained or derived from the patient are obtained or derived from a liquid biopsy. In some embodiments the second state of the cancer of the patient based on identification of a first set of one or more ctDNA molecules from a first plurality of cell-free nucleic acid molecules that is obtained or derived from the patient are obtained or derived from a liquid biopsy. In some embodiments the liquid biopsy is serum, pleural fluid, peritoneal fluid, or stool.

In an aspect, the present disclosure provides a method of monitoring progress of a MSI-H solid tumor of a patient, the method comprising: (a) determining a first state of the tumor of the patient based on identification of a first set of one or more ctDNA molecules from a first plurality of cell-free nucleic acid molecules that is obtained or derived from the patient; (b) determining a second state of the solid tumor of the patient based on identification of a second set of one or more ctDNA molecules from a second plurality of cell-free nucleic acid molecules that is obtained or derived from the patient, wherein the second plurality of cell-free nucleic acid molecules are obtained from the patient subsequent to obtaining the first plurality of cell-free nucleic acid molecules from the patient, wherein the second state of the solid tumor is determined after one or more doses of an immune checkpoint inhibitor; and (c) determining the progress of the solid tumor based on the first state of the solid tumor and the second state of the solid tumor, wherein the second state of the solid comprises a reduction in the level of ctDNA molecules.

In an aspect, the present disclosure provides a method of treating a patient with a MSI-H solid tumor, the method comprising: (a) determining a first state of the MSI-H solid tumor based on identification of a first set of one or more ctDNA molecules from a first plurality of cell-free nucleic acid molecules that is obtained or derived from the patient; (b) determining a second state of the MSI-H solid tumor based on identification of a second set of one or more ctDNA molecules from a second plurality of cell-free nucleic acid molecules that is obtained or derived from the patient, wherein the second plurality of cell-free nucleic acid molecules are obtained from the patient subsequent to obtaining the first plurality of cell-free nucleic acid molecules from the patient, wherein the second state of the MSI-H solid tumor is determined after one or more doses of an immune checkpoint inhibitor; (c) determining the progress of the MSI-H solid tumor based on the first state of the solid tumor and the second state of the solid tumor, wherein the second state of the solid tumor comprises a reduction in the level of ctDNA molecules; and, (d) administering at least one additional dose of the immune checkpoint inhibitor.

In some embodiments, the second plurality of cell-free nucleic acid molecules that is obtained or derived from the patient, wherein the second plurality of cell-free nucleic acid molecules are obtained from a liquid biopsy collected 3, 4, 5, 6, 7, 8, 9, 10, 11, 12, 13, 14, 15, 16, 17, 18, 19, 20, 21, 22, 23, or 24 weeks after the first immune checkpoint inhibitor dose.

In some embodiments, the liquid biopsy is stool, pleural fluid, peritoneal fluid, or serum. In some embodiments the liquid biopsy is serum. In some embodiments the liquid biopsy is pleural fluid. In some embodiments the liquid biopsy is a needle aspiration from a solid tumor. In some embodiments the liquid biopsy is a lung tumor needle biopsy. In some embodiments the liquid biopsy is a transrectal biopsy of the prostate. In some embodiments the liquid biopsy is a transperineal biopsy of the prostate. In some embodiments the liquid biopsy is a bone marrow biopsy.

In some embodiments, the ctDNA level is determined as the mean variant allele fraction (VAF) from a particular patient sample and the Molecular Response (MR) the ratio of the mean VAF measured during a line of therapy or treatment to the baseline VAF. In some embodiments, a MR is calculated at multiple time points while a patient is on treatment.

In some embodiments, the second state of the solid tumor comprises a reduction in the level of ctDNA molecules derived or obtained from a second liquid biopsy, wherein the second liquid biopsy is collected between 3 weeks after the first immune checkpoint inhibitor dose and 24 weeks after the first immune checkpoint inhibitor dose. In some embodiments, the second liquid biopsy is collected 4, 5, 6, 7, 8, 9, 10, 11, 12, 13, 14, 15, 16, 17, 18, 19, 20, 21, 22, 23, or 24 weeks after the first immune checkpoint inhibitor dose. In some embodiments, one or more later states of the solid tumor are determined after the second state of the solid tumor. In some embodiments, the one or more later states of the solid tumor are determined from a liquid biopsy collected from the patient from between 3 and 24 weeks after the collection of the second liquid biopsy.

In an embodiment reduction in the level of ctDNA molecules correlates with the radiographic overall response rate as measured by RECIST guidelines.

In an aspect, the present disclosure provides a method of treating breast cancer, the method comprising selecting a breast cancer patient with a positive baseline circulating tumor DNA (ctDNA) sample; administering a first therapeutic intervention; administering a second therapeutic intervention; and, measuring the ctDNA levels of the patient. In some embodiments, the first therapeutic intervention is surgery, radio therapy, an immune checkpoint inhibitor, or a chemotherapeutic. In some embodiments, the second therapeutic intervention is one or more immune checkpoint inhibitor. In some embodiments, the immune checkpoint inhibitor is a PD-1 antagonist, a TIGIT antagonist, or a combination thereof.

In various embodiments, the method of treating cancer is efficacious as measured by ctDNA detected molecular response. In various embodiments the method of treating cancer is efficacious as measured by ctDNA detected minimal residual disease (MRD).

### PD-1 and PD-L1 Inhibitors

In some embodiments, the immune checkpoint inhibitor is a PD-1 antagonist. In an embodiment, the PD-1 antagonist is an anti-PD-1 antibody. In some embodiments, the anti-PD-1 antibody is selected from the group consisting of pembrolizumab or a biosimilar thereof, nivolumab or a biosimilar thereof, cemiplimab or a biosimilar thereof, and dostarlimab or a biosimilar thereof. In an embodiment, the anti-PD-1 antibody is pembrolizumab or a biosimilar thereof. In an embodiment, the anti-PD-1 antibody is dostarlimab or a biosimilar thereof.

Pembrolizumab is a humanized monoclonal anti-PD-1 antibody commercially available as KEYTRUDA. Pembrolizumab is indicated for the treatment of some unresectable or metastatic melanomas. The amino acid sequence of pembrolizumab and methods of using are disclosed in U.S. Pat. No. 8,168,757. Methods of using pembrolizumab are also disclosed in U.S. Pat. No. 8,728,474.

Nivolumab is a humanized monoclonal anti-PD-1 antibody commercially available as OPDIVO. Nivolumab is indicated for the treatment of some unresectable or metastatic melanomas. Other names for nivolumab include: BMS-936558, MDX-1106, and ONO-4538. The amino acid sequence for nivolumab and methods of using and making are disclosed in U.S. Pat. No. 8,008,449.

Cemiplimab is a human monoclonal anti-PD-1 antibody commercially available as LIBTAYO. Cemiplimab is indicated for the treatment of non-small cell lung cancer (NSCLC), basal cell carcinoma (BCC), and cutaneous squamous cell carcinoma (CSCC).

Dostarlimab is a human monoclonal anti-PD-1 antibody commercially available as JEMPERLI. Dostarlimab is indicated for the treatment of endometrial cancer and MSI-H or dMMR solid tumors. The amino acid sequence for dostarlimab and methods of using are disclosed in U.S. Pat. Nos. 9,815,897, 10,738,117, and 11,407,830.

In an embodiment, the immune checkpoint inhibitor is selected from the group consisting of nivolumab, pembrolizumab, atezolizumab, avelumab, durvalumab, cemiplimab, dostarlimab, vopratelimab (JTX-4014), spartalizumab (PDR001), camrelizumab (SHR1210), sintilimab (IBI308), tislelizumab (BGB-A317), toripalimab (JS 001), INCMGA00012 (MGA012), AMP-224, AMP-514 (MEDI0680), acrixolimab (YBL-006), KN035, cosibelimab (CK-301), AUNP12, and CA-170, BMS-986189, a macrocyclic peptide, a modified oligonuclotide and a small molecule.

### TIGIT Inhibitors

T-cell immunoglobulin and ITIM domain (TIGIT, also known as WUCAM, Vstm3, and Vsig9) is an inhibitory immune checkpoint. TIGIT is expressed on natural killer cells (NK cells), cytotoxic CD8+ T cells, and regulatory T cells (Tregs). TIGIT is a receptor of the Ig superfamily and has a transmembrane protein with a single extra-cellular IgV domain and a cytoplasmic tail. The nectin and nectin-like proteins CD155 (poliovirus receptor, PVR) and CD112 (poliovirus receptor-related 2, PVRL2) expressed in antigen-presenting cells, T cells, and tumor cells bind to TIGIT. In NK cells, TIGIT has a critical role and upon binding of CD155, phosphorylation through Fyn and Lck occurs, leading to the recruitment of SHIP1, which then blocks signal transduction via PI3K and MAPK pathways. In addition, with phosphorylation, the ITT-like motif of the cytoplasmic tail of TIGIT recruits SHIP1 to decrease the NF-kB signaling. In T cells, TIGIT inhibits their proliferation and activation, not only by directly downregulating the expression of T-cell receptor (TCR) alpha chains and other molecules that comprise the TCR complex, but also by reducing the TCR induced phosphorylation of ERK and production of IFNγ. See, e.g., Yu X. et al. “The surface protein TIGIT suppresses T cell activation by promoting the generation of mature immunoregulatory dendritic cells”. Nat Immunol. 2009; 10:48-57; Joller N. et al., “Cutting edge: TIGIT has T cell-intrinsic inhibitory functions.”, J Immunol. 2011; 186:1338-42; and, Chauvin J-M, et al. “TIGIT and PD-1 impair tumor antigen-specific CD8+ T cells in melanoma patients”. J Clin Invest. 2015; 125:2046-58.

TIGIT inhibitors block the function of the TIGIT receptor. Vibostolimab (MK-7684) is a humanized IgG1 monoclonal antibody that binds to and blocks the function of TIGIT. WO2020/096915 and WO2021/021767 provided method of treating cancer with vibostolimab.

In some embodiments, the anti-TIGIT antibody is selected from the group consisting of tiragolumab (MTIG7192A, RG6058 or RO7092284), vibostolimab (MK-76S4), ASP8374 (PTZ-201), EOS884448 (EOS-448), SEA-TGT (SGN-TGT)), BGB-A1217, BMS-986207 (ONO-4686), COM902 (CGEN-15137), IBI939, domvanalimab (AB154), M6223, AB30S, AB154, TJ-T6, MG1131, NB6253, HLX301, HLX53, SL-9258 (TIGIT-Fc-LIGHT), STW264, and YBL-012. In some embodiments, the anti-TIGIT antibody is selected from the group consisting of tiragoiumab (MTIG7192A, RG6058 or RO7092284), vibostolimab (MK-7684), ASP8374 (PTZ-201), EOS-448, and SEA-TGT (SGN-TGT).

In some aspects, the anti-TIGIT antagonist antibody is an antibody having intact Fc-mediated effector function (e.g, tiragolumab, vibostolimab, etigilimab, EOS084448, or TJ-T6) or enhanced effector function (e g., SGN-TGT). In other aspects, the anti-TIGIT antagonist antibody is an antibody that lacks Fc-mediated 5 effector function (e.g., domvanalimab, BMS-986207, ASP8374, or COM902). In some aspects, the anti-TIGIT antagonist antibody is an IgG1 class antibody, e.g., tiragolumab, vibostokmab, domvanakmab, BMS-9S6207, etigilimab, BGB-A1217, SGN-TGT, EOS08444S (EOS-448), TJ-T6, or AB308. In other aspects, the anti-TIGIT antagonist antibody is an IgG4 class antibody, e.g., ASP8374 or 10 COM902. Tiragolumab (RO7092284) is fully human IgG1 an anti-TIGIT antagonist antibody. Domvanalimab (AB-154) is a high affinity humanized IgG1 anti-TIGIT antibody that enables NK-mediated antibody-dependent cell-mediated cytotoxicity. Domvanalimab has been engineered to lack the FcγR-binding function in order to decrease the risk of depleting intratumoral CD8+effector T cells. WO2017/152088 discloses domvanalimab and methods for treating cancer

Other anti-TIGIT antibodies in clinical development include ociperlimab etigilimab, ASP8374 (Shirasuna K. et al., “Characterization of ASP8374, a fully-human, antagonistic anti-TIGIT monoclonal antibody”. Cancer Treat Res Commun 28:100433 (2021)); and, EOS884448 (EOS-448; WO2019/023504).

### Other Immune Check Point Inhibitors

The protein T Cell Immunoglobulin and Mucin Domain-3 (TIM-3), also known as Hepatitis A Virus Cellular Receptor 2 (HAVCR2), is a Th1-specific cell surface protein that regulates macrophage activation and enhances the severity of experimental autoimmune encephalomyelitis in mice. TIM-3 is highly expressed on the surface of multiple immune cell types, including, for example, Th1 IFN-gamma+ cells, Th17 cells, natural killer (NK) cells, monocytes, and tumor-associated dendritic cells (DCs) (see, e.g., Clayton et al., J. Immunol., 192(2): 782-791 (2014); Jones et al., J. Exp. Med., 205:2763-2779 (2008); Baitsch et al., PLOS ONE, 7: e30852 (2012); Ndhlovu et al., Blood, 119:3734-3743 (2012). TIM-3 also is highly expressed on “exhausted” or impaired CD8+ T-cells in a variety of chronic viral infections (e.g., HIV, HCV, and HBV) and in certain cancers (see, e.g., McMahan et al., J. Clin. Invest., 120 (12): 4546-4557 (2010); Jin et al., Proc Natl Acad Sci USA, 107(33): 14733-14738 (2010); Golden-Mason et al., J. Virol., 83(18): 9122-9130 (2009); Jones et al., supra; Fourcade et al., J. Exp. Med., 207(10): 2175-2186(2010); Sakuishi et al., J. Exp. Med., 207(10): 2187-2194 (2010); Zhou et al., Blood, 117(17): 4501-4510 (2011); Ngiow et al., Cancer Res., 71(10): 3540-3551 (2011)). Cobolimab, also known as TSR-022, and described in U.S. Pat. No. 10,508,149, binds to and inhibits the TIM-3 function.

In some embodiments, a method of treatment comprises antagonism of TIM-3 and PD-1, such as in treatment of melanoma, colorectal cancer, AML, hepatitis C virus infection, HBV infection, and lymphocytic choriomeningitis virus infection.

In an embodiment, a method of increasing the efficacy of a DNA-based tumor vaccine comprises TIM-3 blockade by an antibody or small molecule. In an embodiment, a method of increasing responsiveness to cisplatin comprises TIM-3 blockade by an antibody or small molecule.

In an embodiment, a method of inhibiting tumor growth by antibody blockade of TIM-3 and PD-1, such as head and neck cancer.

In an embodiment, a method of detecting a solid tumor or a malignant hematopoietic cell that comprises measuring TIM-3 expression, such as in colon, gastric and cervical cancer, non-small-cell lung cancer and clear cell renal carcinoma, AML cells, and leukemic stem cells.

In an embodiment, a method of treatment of cancer comprises detection of a polymorphism in a coding or a non-coding region of HAVCR2 and treating with a TIM-3 antagonist or an antibody that binds TIM-3. Polymorphisms in HAVCR2 including but not limited to −1516G/T (rs10053538), −574G/T (rs10515746), and +4259T/G (rs1036199) associated with cancer of the gastrointestinal tract, renal cell carcinoma and pancreatic cancer, non-small-cell lung cancer and in patients infected with HIV with non-Hodgkin lymphoma.

In an embodiment, a method of treatment of cancer comprises detection of a polymorphism in the PD1 gene (+8669AA (rs10204525)) or HAVCR2 gene (−1516G/T) and treating with a TIM-3 antagonist or an antibody that binds TIM-3, such as in patients infected with HBV and with hepatocellular carcinoma.

In some embodiments, the ICI is cobolimab. In some embodiments, the ICI is a combination of cobolimab and an anti-PD-1 inhibitor. In some embodiments, the ICI is a combination of cobolimab and pembrolizumab. In some embodiments, the ICI is a combination of cobolimab and dostarlimab.

Encilimab (also known as TSR-033) is a Lymphocyte Activation Gene-3 (LAG-3) binding agent and is disclosed in WO20/18201096. Relatlimab is a commercially available LAG-3 inhibitor and is disclosed in WO2014/008218. In some embodiments, the ICI is a Lag-3 inhibitor. In some embodiments, the Lag-3 inhibitor is encilimab. In some embodiments, the Lag-3 inhibitor is relatlimab. In some embodiments, the ICI is a combination of a PD-1 inhibitor and a Lag-3 inhibitor. In some embodiments, the ICI is a combination of nivolumab and relatlimab.

Aspects of the invention further include:

Use of a pharmaceutical composition comprising an immune checkpoint inhibitor for a treatment of a cancer wherein a change in an amount of circulating tumor DNA is monitored to determine a therapeutic response. The use of a pharmaceutical composition, the use further comprising wherein the change in the amount of circulating tumor DNA is the change between a baseline amount and an amount after at least one dose of the pharmaceutical composition comprising the immune checkpoint inhibitor. The use of a pharmaceutical composition, the use further comprising wherein the change in the amount of circulating tumor DNA is a reduction of at least from about 5% to about 70% and wherein the reduction is determined from a liquid biopsy collected 3, 4, 5, 6, 7, 8, 9, 10, 11, 12, 13, 14, 15, 16, 17, 18, 19, 20, 21, 22, 23, or 24 weeks after the first immune checkpoint inhibitor dose. The use of a pharmaceutical composition, the use further comprising wherein the change in the amount of circulating tumor DNA is a reduction of at least about 15%. The use of a pharmaceutical composition, the use further comprising wherein the change in the amount of circulating tumor DNA is a reduction of at least from about 15% to about 50%.

The use of a pharmaceutical composition, the use further comprising wherein the immune checkpoint inhibitor is a PD-1 inhibitor, a PD-L1 inhibitor, a CTLA-4 inhibitor, a TIGIT inhibitor, a LAG-3 inhibitor, a CD96 inhibitor, or a TIM-3 inhibitor. The use of a pharmaceutical composition, the use further comprising wherein the immune checkpoint inhibitor is a PD-1 inhibitor. The use of a pharmaceutical composition, the use further comprising wherein in the PD-1 inhibitor is pembrolizumab, nivolumab, cemiplimab, or dostarlimab. The use of any one of the preceding claims wherein the treatment comprises radiotherapy.

The use of a pharmaceutical composition, the use further comprising wherein the cancer is non-small cell lung cancer.

Use of circulating tumor DNA to assess cancer progression in a patient receiving immune checkpoint inhibitor therapy.

A method for treating a solid tumor in a human patient, the method comprising administering an immune checkpoint inhibitor to the patient having circulating tumor DNA that is detectable before the first dose of the immune checkpoint inhibitor and a level of circulating tumor DNA is decreased following one or more doses of the immune checkpoint inhibitor by at least from about 5% to about 70%.

The method for treating a solid tumor in a human patient, the method further comprising wherein the immune checkpoint inhibitor is an anti-PD-1 inhibitor, an anti-PD-L1 inhibitor, a CTLA-4 inhibitor, a TIGIT inhibitor, a CD96 inhibitor, a TIM-3 inhibitor, or a combination thereof.

The method for treating a solid tumor in a human patient further comprising wherein the immune checkpoint inhibitor is pembrolizumab, nivolumab, cemiplimab, ipilimumab, dostarlimab, or a biosimilar thereof. The method for treating a solid tumor in a human patient further comprising wherein the immune checkpoint inhibitor is a combination of an anti-PD-1 inhibitor and a TIGIT inhibitor. The method for treating a solid tumor in a human patient further comprising wherein the anti-PD-1 inhibitor is selected from the group consisting of dostarlimab and pembrolizumab and the TIGIT inhibitor is selected from the group consisting of vibostolimab and EOS-448. The method for treating a solid tumor in a human patient further comprising wherein the administering step comprises radiotherapy.

A method of treating cancer in a patient comprising providing an immune checkpoint inhibitor to the patient; detecting circulating tumor DNA in the patient; determining a molecular response rate of the cancer to the immune checkpoint inhibitor; and, determining a radiologic objective response rate.

A method of treating cancer in a patient comprising determining a baseline circulating tumor DNA profile from a liquid biopsy; providing an immune checkpoint inhibitor to the patient; determining a second circulating tumor DNA level from a second liquid biopsy; determining a molecular response rate of the cancer to the immune checkpoint inhibitor based on the step of determining a baseline and the step of determining a second circulating tumor DNA level; and, determining a radiologic objective response rate.

A method of treatment of cancer in a patient previously treated with an immune checkpoint inhibitor comprising the steps of detecting circulating tumor DNA in a liquid biopsy from the patient, determining a molecular response rate, and treating the patient with an immune checkpoint inhibitor.

A method of treatment of cancer comprising dosing a patient with an immune checkpoint inhibitor, detecting circulating tumor DNA after a therapeutic interval, and determining on-treatment change in cancer. The method for treating a solid tumor in a human patient further comprising wherein molecular response rate correlates with a radiologic objective response.

A method of treatment of cancer in a patient comprising the steps of detecting circulating tumor DNA in the patient, providing an immune checkpoint inhibitor to the patient, detecting circulating tumor DNA in the patient after providing the checkpoint inhibitor, and measuring cancer progression. The method for treating a solid tumor in a human patient further comprising, wherein the circulating tumor DNA of one or both detecting steps is obtained from a liquid biopsy from the patient. The method for treating a solid tumor in a human patient further comprising, wherein the circulating tumor DNA of one or both detecting steps is obtained from a cell of the patient. The method for treating a solid tumor in a human patient further comprising, wherein the cancer progression is assessed by a radiologic objective response rate. The method for treating a solid tumor in a human patient further comprising, wherein the cancer progression improved as compared to the cancer progression prior to providing the immune checkpoint inhibitor.

A method of treatment of a cancer in a patient comprising providing a pharmaceutical composition comprising an immune checkpoint inhibitor to the patient, detecting a change in an amount of circulating tumor DNA in the patient, and determining a therapeutic response. The method for treating a solid tumor in a human patient further comprising, wherein the change in the amount of circulating tumor DNA is the change between a baseline amount of circulating tumor DNA isolated from a first liquid biopsy and the amount of circulating tumor DNA isolated from a second liquid biopsy obtained after at least one dose of a pharmaceutical composition comprising an immune checkpoint inhibitor. The method for treating a solid tumor in a human patient further comprising wherein the change in the amount of circulating tumor DNA is a reduction of at least from about 5% to about 70% and wherein the second liquid biopsy collected 3, 4, 5, 6, 7, 8, 9, 10, 11, 12, 13, 14, 15, 16, 17, 18, 19, 20, 21, 22, 23, or 24 weeks after the first immune checkpoint inhibitor dose. The method for treating a solid tumor in a human patient further comprising wherein the change in the amount of circulating tumor DNA is a reduction of at least from about 15% to about 50%. The method for treating a solid tumor in a human patient further comprising wherein the change in the amount of circulating tumor DNA is a reduction of at least about 15%. The method for treating a solid tumor in a human patient further comprising wherein the immune checkpoint inhibitor is a PD-1 inhibitor, a PD-L1 inhibitor, a CTLA-4 inhibitor, a TIGIT inhibitor, a LAG-3 inhibitor, a CD96 inhibitor, or a TIM-3 inhibitor. The method for treating a solid tumor in a human patient further comprising wherein the immune checkpoint inhibitor is a PD-1 inhibitor. The method for treating a solid tumor in a human patient further comprising wherein in the PD-1 inhibitor is pembrolizumab, nivolumab, cemiplimab, or dostarlimab. The method for treating a solid tumor in a human patient further comprising, wherein the immune checkpoint inhibitor is atezolizumab, ipilimumab, avelumab, durvalumab, vopratelimab, spartalizumab, camrelizumab, sintilimab, tislelizumab, toripalimab, INCMGA00012, AMP-224, AMP-514, acrixolimab, KN035, cosibelimab, AUNP12, CA-170, or BMS-986189. The method for treating a solid tumor in a human patient further comprising wherein the cancer is non-small cell lung cancer.

A method of treatment of a cancer in a patient previously treated with an immune checkpoint inhibitor comprising detecting circulating tumor DNA in the patient, determining a molecular response rate, and treating the patient with an immune checkpoint inhibitor.

A method of treatment of a cancer in a patient comprising obtaining a sample from a patient, isolating a biomarker form the sample, and predicting response of the patient response to an immune checkpoint inhibitor. The method for treating a solid tumor in a human patient further comprising wherein the biomarker is selected from PDL-1 expression, hypoxia, tumor mutation burden, interferon-γ, extracellular matrix, gene expression, microbiome, promoter hypermethylation, and molecular or cellular characterization within the tumor microenvironment.

A method of treatment of a cancer in a patient comprising providing a pharmaceutical composition comprising an immune checkpoint inhibitor to the patient, detecting a change in an amount of circulating tumor DNA in the patient, and determining a therapeutic response.

A method of treatment of a cancer in a patient comprising providing a first pharmaceutical composition comprising an immune checkpoint inhibitor to the patient, detecting a change in an amount of circulating tumor DNA in the patient, determining a therapeutic response, and providing a second pharmaceutical composition comprising an immune checkpoint inhibitor to the patient. The method for treating a solid tumor in a human patient further comprising wherein the first composition and the second composition contain different immune checkpoint inhibitors. The method for treating a solid tumor in a human patient further comprising wherein the first composition and the second composition contain different amounts of immune checkpoint inhibitors.

A method of treatment of a disease in a patient comprising providing a pharmaceutical composition comprising an immune checkpoint inhibitor to the patient, detecting a change in an amount of circulating tumor DNA in the patient, and determining a therapeutic response.

### EXAMPLES

### Example 1

### Dataset for Meta Analysis

MRR and ORR information was retrieved from eight studies/arms1-5 where metastatic solid tumors were treated by ICI. Patient level MR and OS data were downloaded from the report6 (Thompson set as reported JCO Precision Oncology no. 5 (2021) 510-524) or extracted by digitizing published Kaplan-Meier plots using Guyot algorithm1 (MYSTIC set as reported Peters et al., ESMO 2021 Poster 1264P; NCT02453282).

### Simulation Methods

1. Resampling approach: assume a study of two arms with the same sample size per arm (N), the MRR in one arm (control arm) is x, and the MRR in another arm (exp arm) is x+dMRR. The exact number of MR and molecular non-responder (MNR) per arm was calculated and was sampled with replacement from the specified Set, and the hazard ratio between exp and control arms was calculated. The process is repeated 500 times for a given parameter set.

2. Parametric approach: for each dataset (Set1 or Set2), two subpopulations (MRs and MNRs) were identified and piecewise exponential curves were fit to survival data respectively using the R function pch::pchreg with default settings. Censoring data was obtained in the same manner to parametrically sample censoring times. To confirm the adequacy of our simulation strategy (for both failure and censoring time), Kaplan-Meier curves from source data sets were graphically compared to corresponding curves fit to data simulated from corresponding fitted piecewise-exponential distributions. For a given parameter set where the number of MR and MNR in both exp and control arms could be calculated, MRs and MNRs were simulated using the R function PWEALL::rpwe with respective distributions and mixed. This process was repeated 1000 times per a parameter set.

3. Parameter sets: N∈[250,300], x∈[0.2, 0.25, 0.3, 0.35, 0.4] and dMRR∈[0.1, 0.2, 0.3, 0.4]

### Statistical Analysis

Weighted least squares linear regression method was used to study the relationship between MRR and ORR. Hazard ratio for OS was estimated using Cox model. Unless specified, all analyses were performed using R (version 4.1.2)

### Results

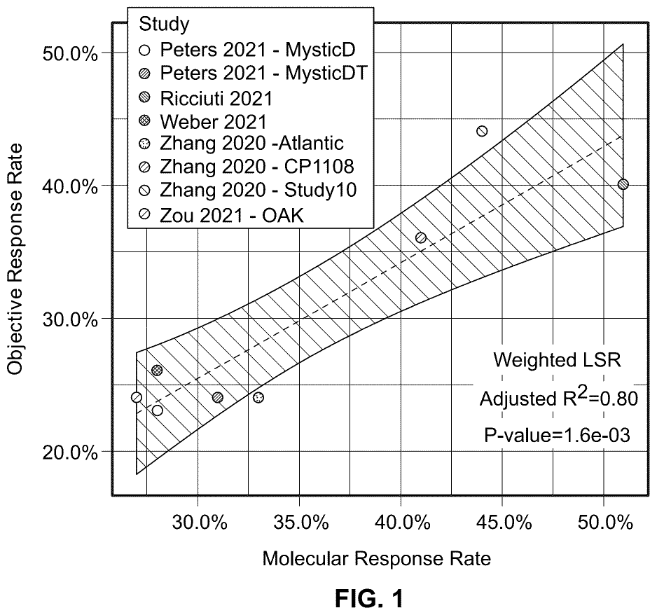

MRR is correlated with ORR in ICI when combined and considered in a meta analysis. MRR shows a linear correlation with ORR in a meta-analysis of clinical trials including ICIs used as front- or second-line therapy. In addition, MRR is numerically similar to ORR, suggesting MRR might hold the potential to be an early proxy for ORR. FIG. 1 shows the least squired regression (LSR) analysis, Weighted LSR Adjusted R²=0.80 and P-value=1.6e⁻⁰³.

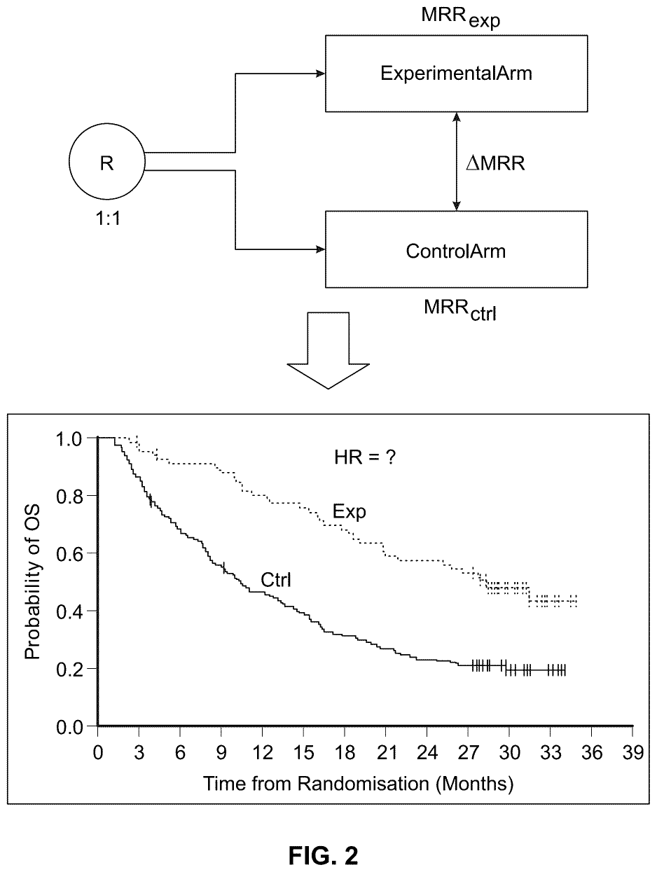

The relationship between ORR and MRR was further analyzed using a hypothetical framework. In a hypothetical framework, two randomized arms are treated by different ICI agents or combinations, and early ctDNA MR is observed at MRRctrl for the control arm, and MRRexp for the experiment arm. The objective is to use simulation to study whether MRR difference between two arms (AMRR) is correlated with difference in overall survival measured by hazard ratio (HR). FIG. 2 shows the hypothetical framework scheme.

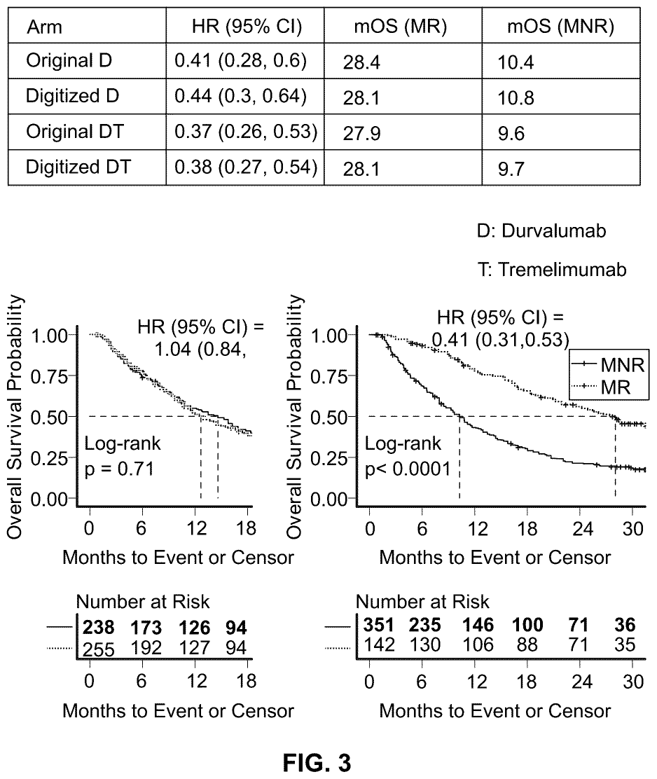

Clinical data from the MYSTIC study was digitized patient-level overall survival data resembles the original data in multiple key aspects. D: durvalumab; DT: durvalumab+tremelimumb (B) D and DT arms of the digitized MYSTIC set are similar in overall survival, and are thus combined in downstream analysis. (C) MRs are significantly associated with overall survival in digitized MYSTIC data (HR=0.41, D and DT arms combined). These data are shown in FIG. 3.

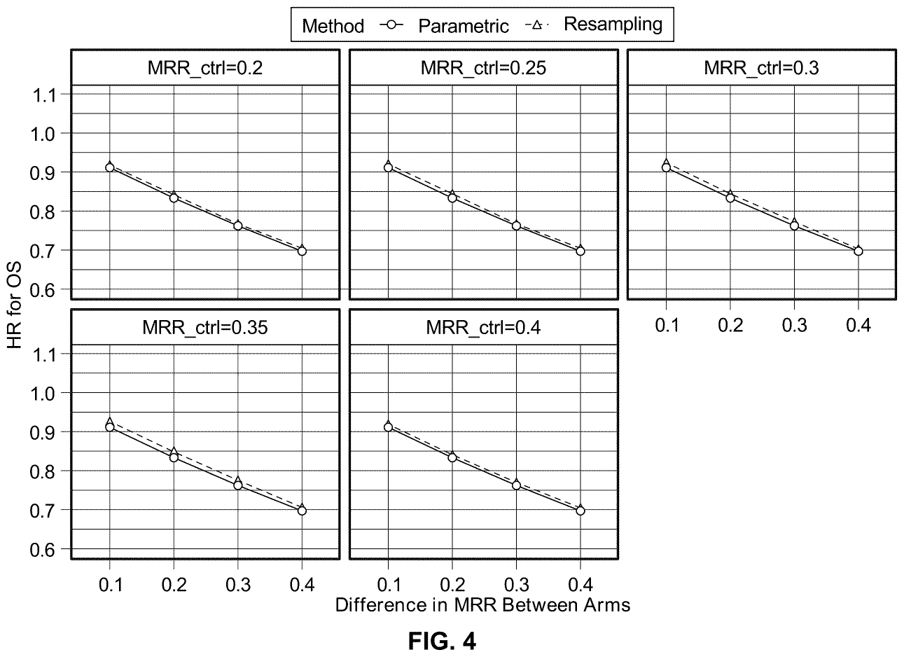

From the analysis, ΔMRR inversely correlated with OS hazard ratio in simulations using digitized MYSTIC data. Greater AMRR correlates with smaller HR between two arms in simulations using MYSITC data. The negative correlation is independent of MRR of the control arm. Highly comparable results are observed from two different simulation methods. FIG. 4 shows these results.

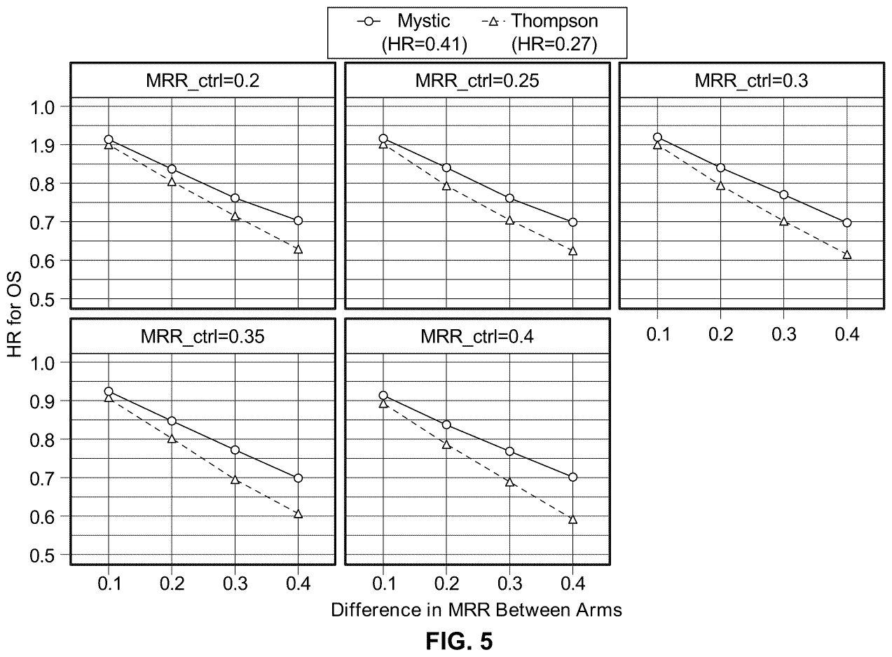

Greater AMRR correlates with smaller HR between two arms in simulations using Thompson data (blue). See FIG. 5. Compared to simulation result from MYSTIC (red), the correlation is stronger due to that fact that MRs are better correlated with OS (HR=0.27). Only result from the resampling approach has been showed here, and higher similar result observed from the parametric approach.

MRR shows a significant correlation with ORR in a meta-analysis of published studies from patients treated with immune checkpoint inhibitors, supporting the further exploration of MRR as a potential surrogate of ORR for early decision making

Simulations in two mNSCLC datasets suggested AMRR between two study arms are inversely correlated with hazard ratio of overall survival, highlighting AMRR may serve as an early proxy of difference in long term survival in randomized studies

Based on these results, measured AMRR predicts hazard ratios and MR is correlated with long term survival and is useful as methods for treating cancer using immune checkpoint inhibitors.

References: Peters et al. ESMO 2021; Ricciuti et al. J Immunother Cancer 2021; Weber et al. JCO Precis Oncol 2021; Zhang et al. Cancer Discov 2020; Zou et al. JCO Precis Oncol 2021; Thompson et al. JCO Precis Oncol 2021.

### Example 2

### Circulating Tumor DNA Profiling of Patients Undergoing Cancer Treatment

Two groups of patients will be studied. One group includes treatment-naïve metastatic patients with non-small cell lung cancer (NSCLC), the other, previously untreated patients with locally advanced, metastatic, PDL-1-selected NSCLC. Both patient groups are screened for treatment with a combination regimen of anti-TIGIT monoclonal antibody and anti-PD-1 monoclonal antibody.

Following screening, these patients will be intravenously administered a coformulated, combination 200 mg of pembrolizumab and 200 mg of vibostolimab on a first day of each cycle. Each cycle commenced will be 3 weeks in length, with up to 35 cycles over about 2 years. This regimen will continue until progression, intolerable adverse events, or a participant and physician decision to cease therapy. Following this combination regimen, the efficacy of the therapy may be assessed by an oncologist. One measure of efficacy is the objective response rate (ORR), which represents the proportion of patients with a reduction in their tumor burden. One important aspect of this measure is that it requires a relatively small population and can be assessed reasonably early as compared with survival trials. While ORR is a typical outcome measure, as a surrogate, analysis of circulating tumor DNA will be carried out with the aim of achieving a simpler and earlier outcome measure that correlates with ORR.

Prior to combination treatment with pembrolizumab/vibostolimab, circulating tumor DNA samples are collected from each patient. The DNA amount is measured to set a baseline level for each patient.

All patients are then treated with the foregoing regimen. Two weeks following treatment, samples of circulating tumor DNA are again collected from each patient and the amount of such DNA is measured to observe any change. After each round of treatment, samples of circulating tumor DNA are collected and measured. Trends toward lowered amounts of such DNA as compared to baseline or any prior measurement indicate a positive therapeutic response and establish efficacy of this treatment regimen.

To validate this treatment outcome measure based on circulating tumor DNA, a side-by-side, statistical analysis is performed. A statistical correlation is established.

### Example 3

Longitudinal Evaluation of Circulating Tumor DNA (ctDNA) in Early Breast Cancer

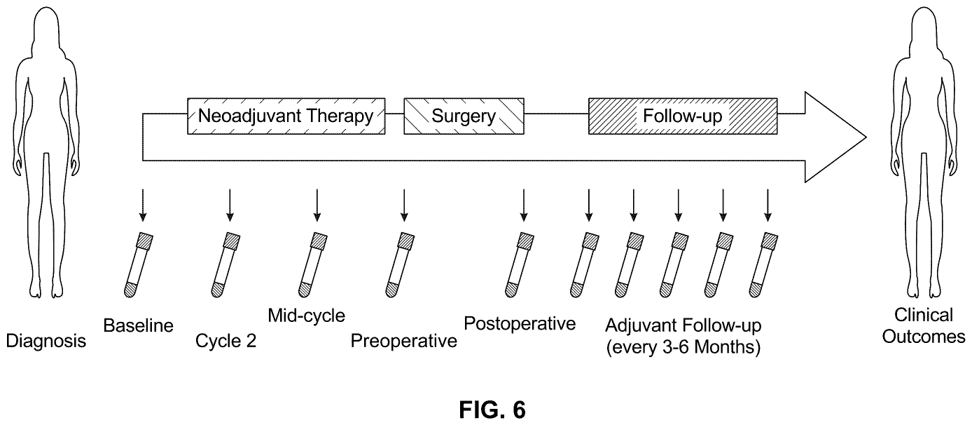

In this study (NCT03702309), patients with early breast cancer (EBC) of all receptor subtypes receiving standard-of-care neoadjuvant therapy were enrolled in a prospective cohort during a roughly sixty-four month period. Plasma samples were collected at baseline, during treatment, perioperatively, and during follow-up, as illustrated in the schematic in FIG. 6.

Each of these plasma samples were analyzed with the Guardant Reveal for Research Use Only assay on the INFINITY platform. ctDNA status (positivity/detected versus negative/not detected) as well as mutational status were detected and reported by based on the assay's predefined thresholds and metrics. ctDNA positivity was defined by a quantified methylation score using differentially methylated regions according to the methods described in Chen S, et al. Cancer Res. 2022; 82:3763-3763, and Greenwald W W, et al. Cancer Res. 2022; 82:3758-3758. Guardant INFINITY detects somatic gene alterations (mutations) in a panel of more than 800 pre-defined genes. For each subject, treatments, outcomes, clinical and pathological characteristics were collected.

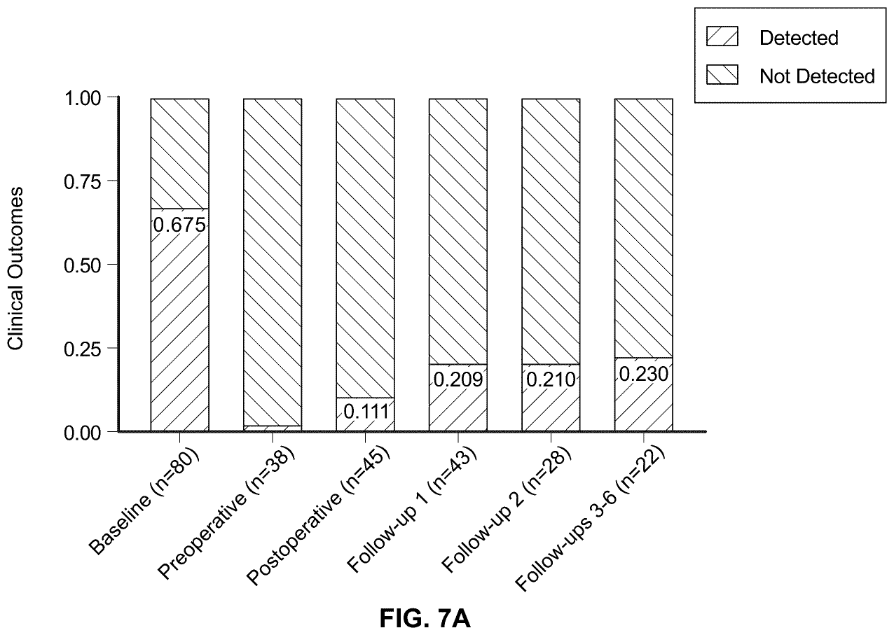

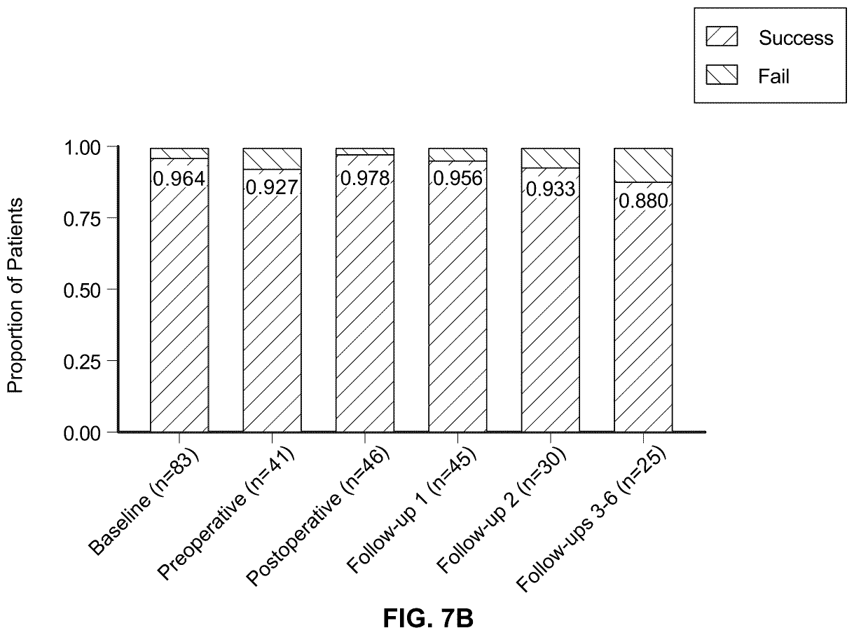

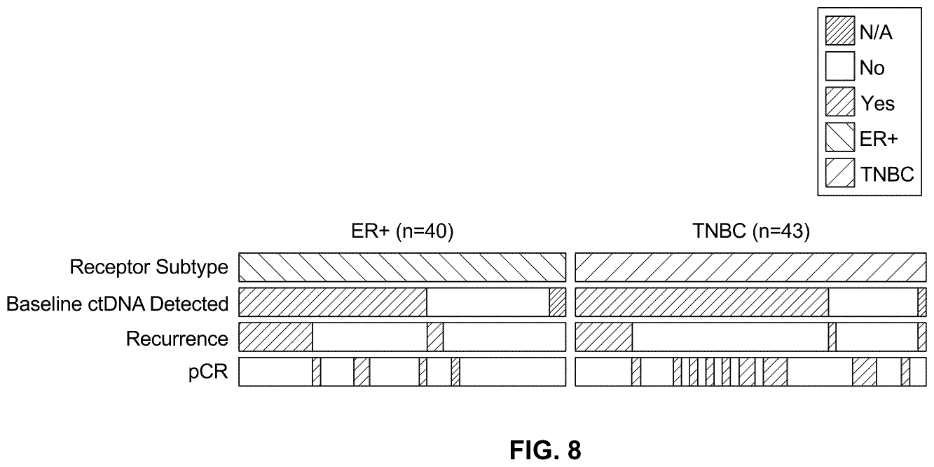

83 participants with early ER+disease (n=40) and TNBC (n=43). FIG. 8 and Table 1 show the overall subject characteristics. From those subjects, 270 samples (median of 3 per participant; with a range of 1-9 per participant) were analyzed in the Guardant INFINITY assay. The median clinical follow-up was 3.7 years with a range of 0.3 to 6.5 years. Results were reported for 95% of the collected serum samples (256 out of 270 samples). The most common reason for no assay results (an assay technical failure) was too little cell-free DNA (cfDNA) being isolated (typically <1 ng total cfDNA). 20 subjects had a clinical recurrence by the data cutoff date. ctDNA was detected at baseline in 67.5% (54 of 80) of the subjects. The ctDNA detection rate for participants with ER+EBC and TNBC were 60.5% (23 of 38) and 73.8% (31 of 42). See FIG. 7A showing ctDNA detection by timepoint and FIG. 7B showing ctDNA isolation and assay success/failure by timepoint. Only three subjects with no ctDNA detected at baseline did not have any ctDNA detected at any later timepoint. Nine participants had a mutation identified at baseline from the INFINITY gene panel: 7 with PIK3CA mutations; and, one subject each for mutations in TP53, FGFR1, BRAF, GATA3, and NOTCH2.

TABLE 1

| | All | | | | | |
|---|---|---|---|---|---|---|
| | Participants | | ER+ | | TNBC | |
| | (n = 83) | | (n = 40) | | (n = 43) | |
| Age, median (range), years | 49 | (24-77) | 49 | (32-77) | 49 | (24-70) |
| Premenopausal, n (%) | 54 | (65.1) | 27 | (67.5) | 27 | (62.8) |
| Postmenopausal, n (%) | 29 | (34.9) | 12* | (30.0) | 16 | (37.2) |
| Taxane, n (%) | 6 | (7.2) | 1 | (2.5) | 5 | (11.6) |
| Anthracycline + taxane, n | 74 | (89.2) | 36 | (90.0) | 38 | (88.4) |
| (%) | | | | | | |
| Pathologic complete | 19 | (22.9) | 5 | (12.5) | 14 | (32.6) |
| response, n (%) | | | | | | |
| Disease stage, n (%) | | | | | | |
| T½ | 52 | (62.7) | 20 | (50.0) | 32 | (74.4) |
| T¾ | 31 | (37.3) | 20 | (50.0) | 11 | (25.6) |
| Nodal status, n (%) | | | | | | |
| N0 | 42 | (50.1) | 16 | (40.0) | 26 | (60.4) |
| N1 | 41 | (49.9) | 24 | (60.0) | 17 | (39.6) |
| Recurrences, n (%) | 20 | (24.1) | 11 | (27.5) | 9 | (20.9) |

Table 2 shows the characteristics of subject who eventually had a clinical recurrence of disease. Median lead time was 115 days with a range of 13-1303 days. Lead time was calculated retroactively from the date of clinical recurrence to the first positive test after definitive therapy (postoperative or in follow-up, whichever is earlier). Legend: **No follow-up samples collected; \hat{ }\hat{ }No previous negative follow-up samples; #Most proximal follow-up sample was negative; †All follow-up samples failed sequencing. The majority of the recurrences were distant recurrences. Lead time refers to the time between a positive ctDNA test and detectable clinical recurrence.

TABLE 2

| | | | | | | Last |
|---|---|---|---|---|---|---|
| Participant | | ctDNA | Methylation | | Lead | Negative |
| Identification | Receptor | Outcome | Score | Recurrence | Time | Sample |
| Number | Subtype | (Baseline) | (Baseline) | Location | (Days) | (Days) |
| 0011 | ER+ | Detected | 0.00709059 | Local | ** | ** |
| 0020 | ER+ | Not | N/A | Distant | 1303 | 1492 |
| | | Detected | | | | |
| 0067 | ER+ | Detected | 0.0124347 | Distant | 13 | 289 |
| 0076 | ER+ | Detected | 0.0119603 | Distant | 78 | \hat{ }\hat{ } |
| 0088 | ER+ | Detected | 0.0519528 | Distant | 28 | 475 |
| 0089 | ER+ | Detected | 0.00060607 | Distant | ** | ** |
| 0116 | ER+ | Detected | 0.00270626 | Distant | 748 | 573 |
| 0122 | ER+ | Detected | 0.00174683 | Distant | ** | ** |
| 0127 | ER+ | Detected | 0.0181849 | Distant | 672 | \hat{ }\hat{ } |
| 0155 | ER+ | Not | N/A | Distant | 152 | \hat{ }\hat{ } |
| | | Detected | | | | |
| 0160 | ER+ | Detected | 0.0934043 | Distant | 15 | \hat{ }\hat{ } |
| 0004 | TNBC | Fail | N/A | Local | † | † |
| 0085 | TNBC | Detected | 0.0423625 | Distant | ** | ** |
| 0093 | TNBC | Detected | 0.00379299 | Distant | ** | ** |
| 0113 | TNBC | Detected | 0.00219049 | Distant | 0 | 417 |
| 0121 | TNBC | Detected | 0.00064337 | Local | # | 328 |
| 0128 | TNBC | Not | N/A | Distant | 521 | \hat{ }\hat{ } |
| | | Detected | | | | |
| 0146 | TNBC | Detected | 0.00134858 | Local | # | 12 |
| 0151 | TNBC | Detected | 0.0492011 | Distant | ** | ** |
| 0166 | TNBC | Detected | 0.0166702 | Distant | # | 334 |

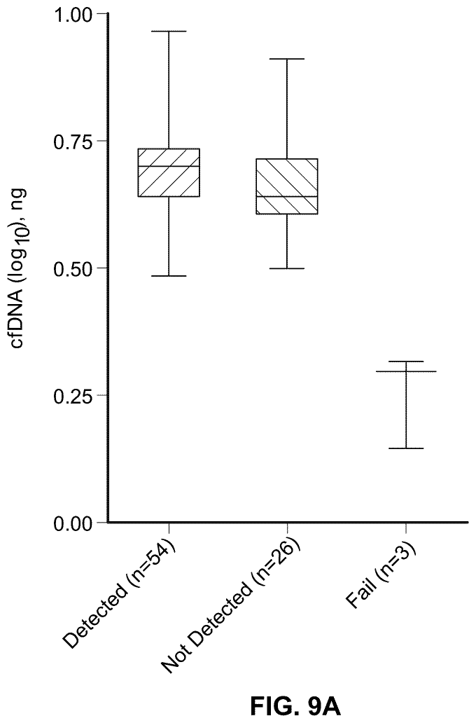

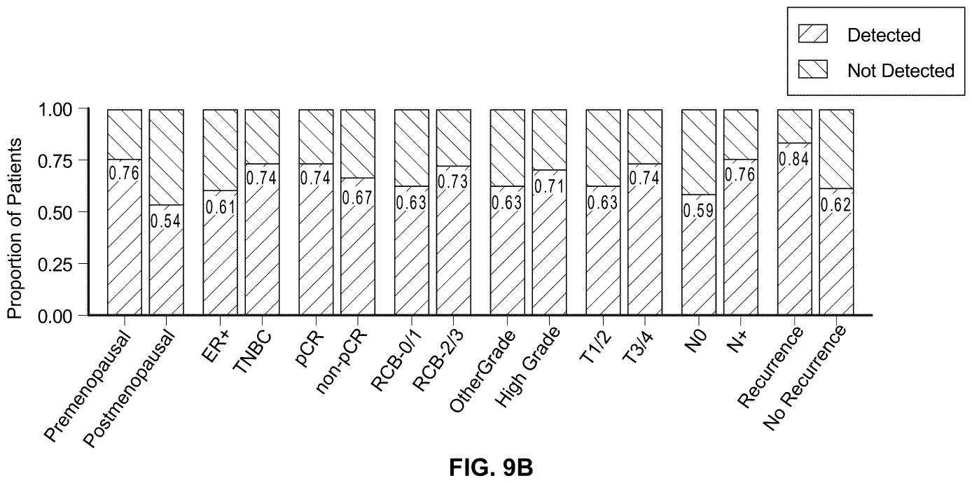

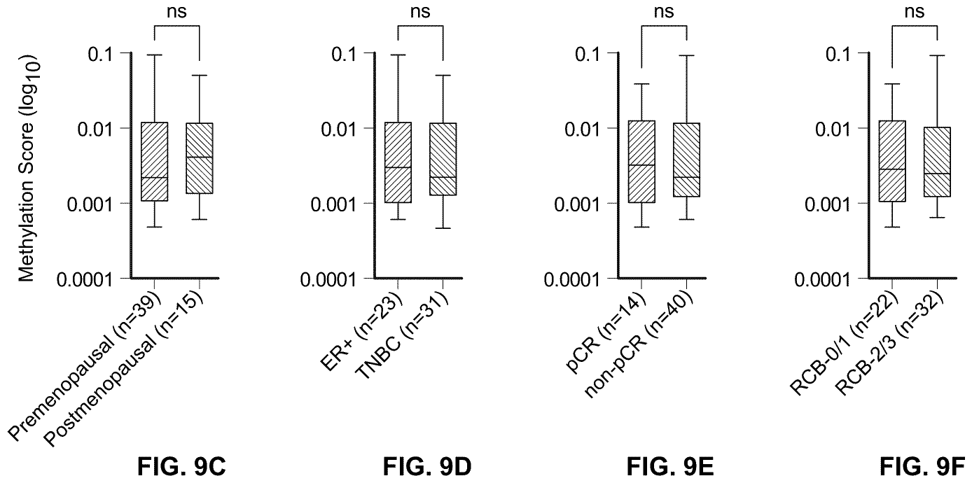

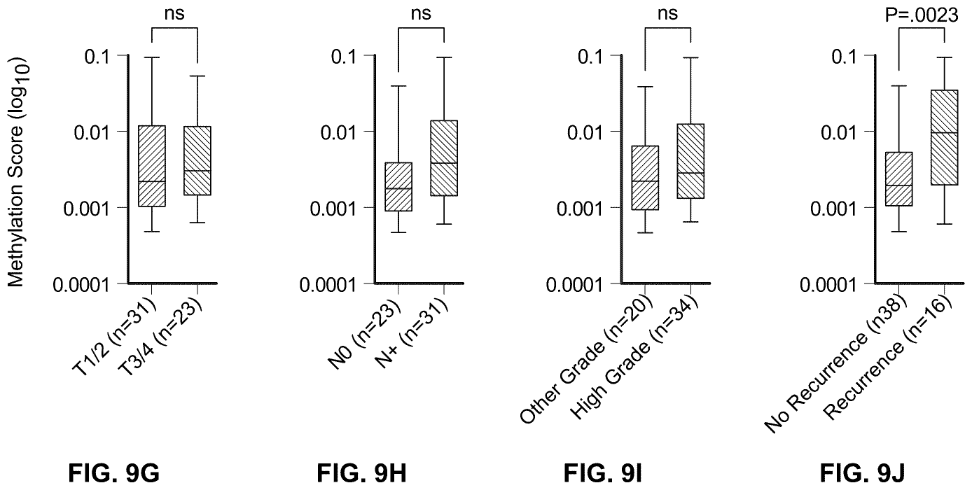

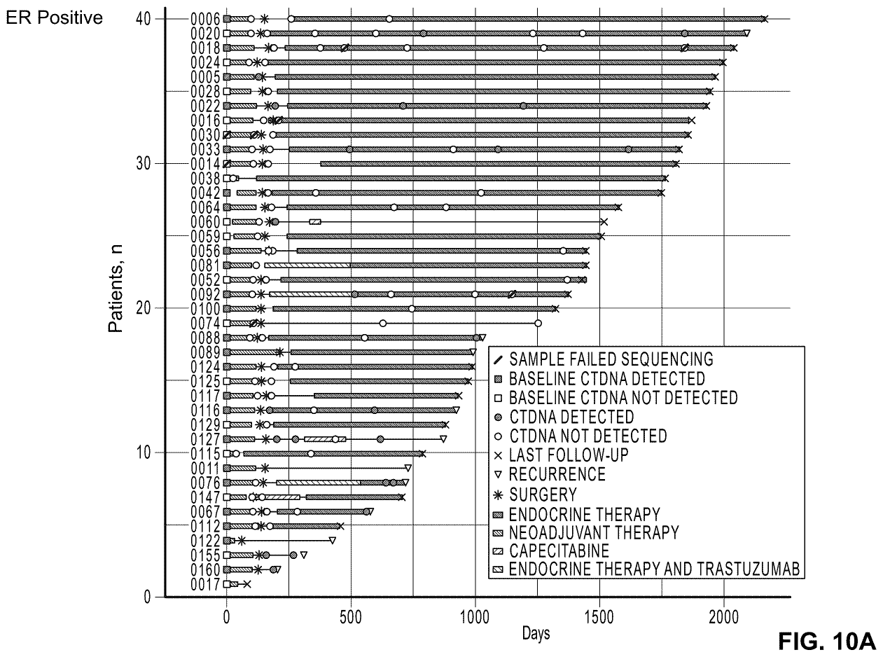

FIG. 9 shows the association of baseline clinical variables with ctDNA detection.

FIG. 9A shows the quantitative cfDNA and associated baseline detection outcome. FIG. 9B shows the proportion of baseline samples with detected ctDNA by selected demographic and clinical characteristics. FIGS. 9C-9J shows box/whiskers plots of the quantitative methylation score by selected demographic and clinical characteristics. The box represents the 25th to 75th percentiles, while the whiskers represent the minimum and maximum values.

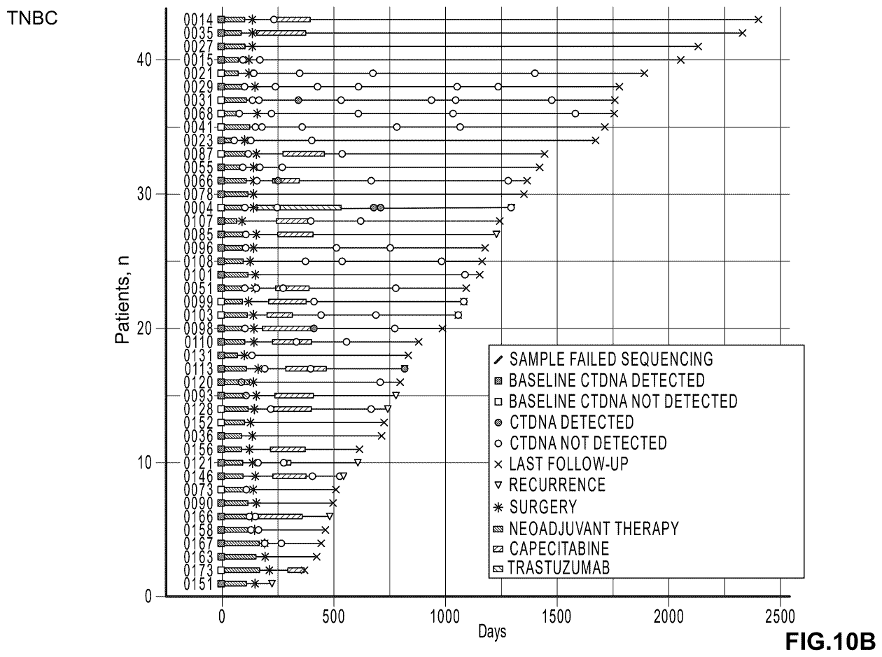

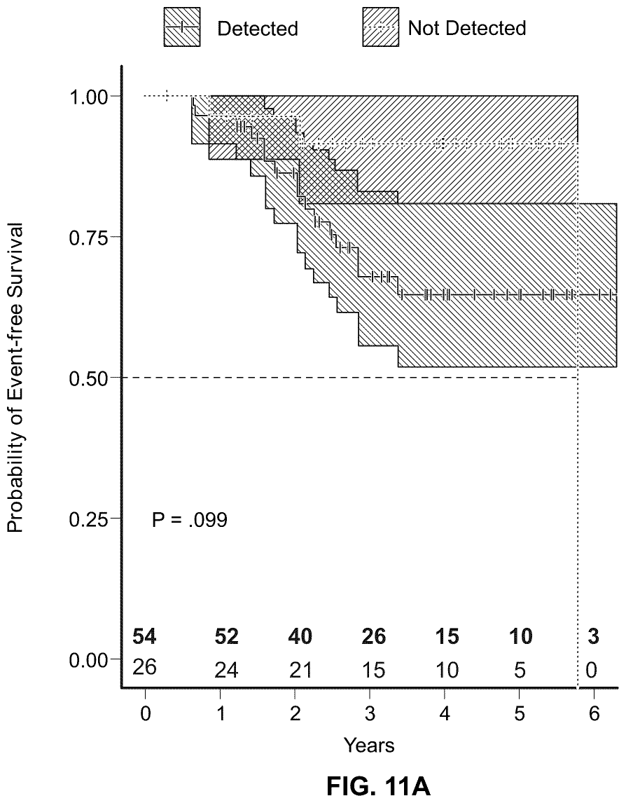

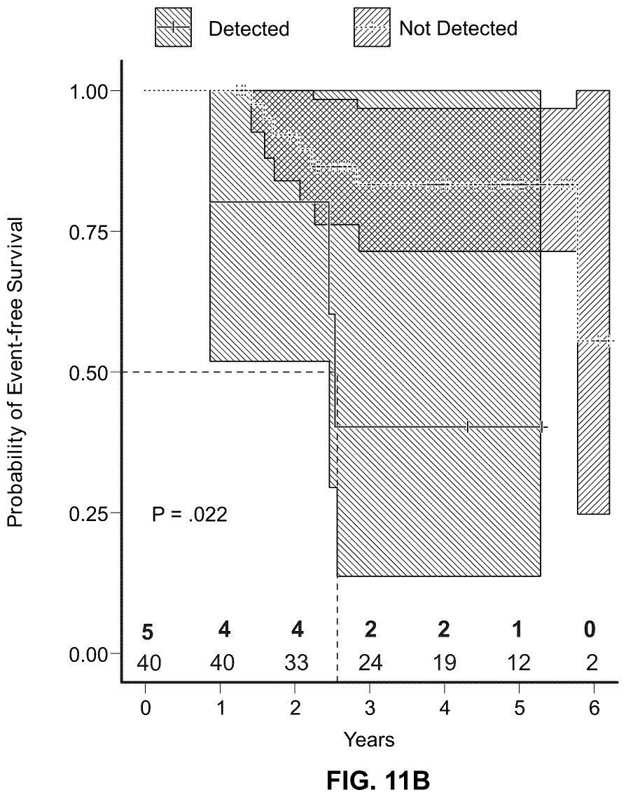

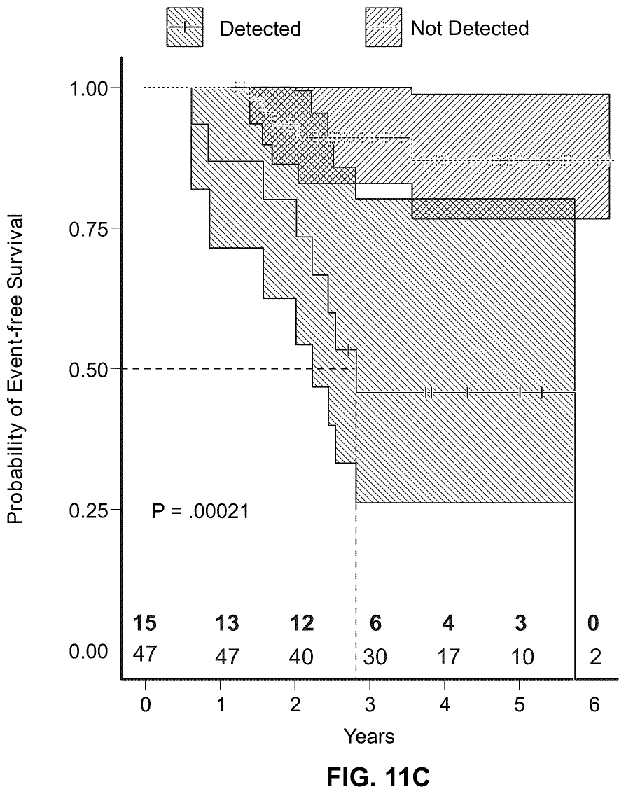

FIGS. 10A and 10B show the clinical timelines for each participant showing the detection of ctDNA and various clinical events, e.g., surgery, recurrence, last follow-up. The bars are coded for treatment: endocrine therapy, neoadjuvant therapy, capecitabine, ir endocrine therapy with trastuzumab. FIG. 10A shows the ER+subjects while FIG. 10B shows the TNBC subjects. Finally, FIGS. 11A-11C shows the exploratory outcome by time of first ctDNA identification (A, baseline ctDNA detected; B, postoperative ctDNA detected; and, C, ctDNA detected postoperative or at later follow-up time points). The numbers along the bottom of the plots shows the number of patients in each cohort (ctDNA detected, ctDNA not detected) at each time point (year 0-6).

The baseline ctDNA methylation score was higher in subjects with eventual clinical recurrence. Furthermore, the detection of methylated ctDNA at the postoperative or the follow-up time points correlated with clinical recurrence.

1. Use of a pharmaceutical composition comprising an immune checkpoint inhibitor for a treatment of a cancer wherein a change in an amount of circulating tumor DNA is monitored to determine a therapeutic response wherein the change in the amount of circulating tumor DNA is the change between a baseline amount and an amount after at least one dose of the pharmaceutical composition comprising the immune checkpoint inhibitor; and, wherein the change in the amount of circulating tumor DNA is a reduction of at least from about 5% to about 70% and wherein the reduction is determined from a liquid biopsy collected 3, 4, 5, 6, 7, 8, 9, 10, 11, 12, 13, 14, 15, 16, 17, 18, 19, 20, 21, 22, 23, or 24 weeks after the first immune checkpoint inhibitor dose, wherein the treatment comprises radiotherapy.

2. The use of claim 1 wherein the change in the amount of circulating tumor DNA is a reduction of at least about 15%.

3. The use of claim 2 wherein the change in the amount of circulating tumor DNA is a reduction of at least from about 15% to about 50%.

4. The use of claim 1 wherein the immune checkpoint inhibitor is a PD-1 inhibitor, a PD-L1 inhibitor, a CTLA-4 inhibitor, a CD96 inhibitor, or a TIM-3 inhibitor.

5. The use of claim 1 wherein the immune checkpoint inhibitor is a PD-1 inhibitor.

6. The use of claim 5 wherein the PD-1 inhibitor is pembrolizumab, nivolumab, cemiplimab, or dostarlimab.

7. (canceled)

8. The use of claim 1 wherein the cancer is non-small cell lung cancer.

9. A method for treating a solid tumor in a human patient, the method comprising administering an immune checkpoint inhibitor to the patient having circulating tumor DNA that is detectable before a first dose of the immune checkpoint inhibitor and a level of circulating tumor DNA is decreased following one or more doses of the immune checkpoint inhibitor by at least from about 5% to about 70%; wherein the immune checkpoint inhibitor is a combination of an anti-PD-1 inhibitor, and a CD96-inhibitor.

10. The method of claim 9 wherein the immune checkpoint inhibitor is pembrolizumab, nivolumab, cemiplimab, dostarlimab, or a biosimilar thereof.

11. (canceled)

12. The method of claim 9 wherein the anti-PD-1 inhibitor is dostarlimab.

13. (canceled)

14. The method for treating a solid tumor in a human patient of claim 9 comprising detecting circulating tumor DNA in the patient, determining a molecular response rate of the cancer to the immune checkpoint inhibitor, and determining a radiologic objective response rate.

15-16. (canceled)

17. A method of treatment of cancer comprising dosing a patient with an immune checkpoint inhibitor, detecting circulating tumor DNA after a therapeutic interval, and determining on-treatment change in cancer.

18. The method of claim 14, wherein molecular response rate correlates with a radiologic objective response.

19-30. (canceled)

31. The method of treatment of cancer of claim 17 further comprising determining a molecular response rate, and treating the patient with an immune checkpoint inhibitor.

32. A method of treatment of a cancer in a patient comprising obtaining a sample from a patient, isolating a biomarker from the sample, and predicting response of the patient to an immune checkpoint inhibitor.

33-34. (canceled)

35. A method of treatment of a cancer in a patient comprising providing a first pharmaceutical composition comprising an immune checkpoint inhibitor to the patient, detecting a change in an amount of circulating tumor DNA in the patient, determining a therapeutic response, and providing a second pharmaceutical composition comprising an immune checkpoint inhibitor to the patient.

36. The method of claim 35 wherein the first pharmaceutical composition and the second pharmaceutical composition contain different immune checkpoint inhibitors.

37. The method of claim 36 wherein the first pharmaceutical composition and the second pharmaceutical composition contain different amounts of immune checkpoint inhibitors.

38. A method of treating breast cancer in a patient, the method comprising: (i) collecting a baseline ctDNA specimen and detecting the baseline level of methylated ctDNA; (ii) administering one or more checkpoint inhibitors; (iii) collecting at least one ctDNA specimen and detecting the level of methylated ctDNA following the administration of one or more checkpoint inhibitors; (iv) determining therapeutic efficacy based on a lower level of detected level of ctDNA.

39. The use of claim 6 wherein the PD-1 inhibitor is dostarlimab.
